(19) United States
(12) Patent Application Publication
(10) Pub. No.: US 2023/0210409 A1
(43) Pub. Date: Jul. 6, 2023

(54) MINIATURIZED WEARABLE DEVICES FOR ANALYTE MEASUREMENT

(71) Applicant: DexCom, Inc., San Diego, CA (US)

(72) Inventors: John Charles Barry, San Diego, CA (US); Elshad Abdullayev, Poway, CA (US); Eunsook Chae Barber, San Diego, CA (US); Patrick J. Castagna, San Diego, CA (US); John Durham, Escondido, CA (US); Craig Thomas Gadd, Lemon Grove, CA (US); Carl E. Hoffmeier, Solana Beach, CA (US); Nicholas Kalfas, San Diego, CA (US); Mark Kempkey, Vista, CA (US); Young Woo Lee, San Diego, CA (US); Nicolas Medjo, San Diego, CA (US); Carl Pettersen, San Diego, CA (US); Will Reyna, San Diego, CA (US); Morgan Alexander Robinson, San Diego, CA (US); Samuel Rogers, San Diego, CA (US); Jeffrey J. Smith, San Diego, CA (US); Terry Thom, Coral Springs, FL (US); Shanger Wang, San Diego, CA (US); James Woodward, San Diego, CA (US)

(21) Appl. No.: 18/091,834

(22) Filed: Dec. 30, 2022

Related U.S. Application Data
(60) Provisional application No. 63/295,819, filed on Dec. 31, 2021.

(51) Int. Cl. A61B 5/145; A61B 5/00

(52) U.S. Cl. CPC A61B 5/14532; A61B 5/6848; A61B 5/6833; A61B 5/742

(57) ABSTRACT

Implementations relate generally to devices for measuring an analyte in a host. Implementations may provide reduced sizes for wearable devices including a transcutaneous analyte sensor for analyte measurement.

### CROSS REFERENCE TO RELATED APPLICATIONS

The present application claims priority to U.S. Provisional Patent Application No. 63/295,819, filed Dec. 31, 2021, the entire contents of which are incorporated herein by reference.

### BACKGROUND

### Field

Systems, methods, and devices for measuring an analyte in an individual are provided. More particularly, on-skin medical devices wearable by a host and having an analyte sensor are provided.

### Description of the Related Technology

Diabetes mellitus is a disorder in which the pancreas cannot create sufficient insulin (Type I or insulin dependent) and/or in which insulin is not effective (Type 2 or non-insulin dependent). In the diabetic state, the victim suffers from high blood sugar, which can cause an array of physiological derangements associated with the deterioration of small blood vessels, for example, kidney failure, skin ulcers, or bleeding into the vitreous of the eye. A hypoglycemic reaction (low blood sugar) can be induced by an inadvertent overdose of insulin, or after a normal dose of insulin or glucose-lowering agent accompanied by extraordinary exercise or insufficient food intake.

Conventionally, a person with diabetes carries a self-monitoring blood glucose (SMBG) monitor, which typically requires uncomfortable finger pricking methods. Due to the lack of comfort and convenience, a person with diabetes normally only measures his or her glucose levels two to four times per day. Unfortunately, such time intervals are spread so far apart that the person with diabetes likely finds out too late of a hyperglycemic or hypoglycemic condition, sometimes incurring dangerous side effects. Glucose levels may be alternatively monitored continuously by a measurement system including an on-skin sensor assembly. The sensor assembly may have a wireless transmitter which transmits measurement data to a receiver which can process and display information based on the measurements.

Minimizing the size of the on-skin sensor assembly may be important in providing a comfortable, minimally intrusive, and user-friendly measurement system. Such a reduced size may increase comfort experienced by a host, among other benefits.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

In a first aspect, an on-skin wearable medical device configured to be deployed to a skin of a host, the on-skin wearable medical device comprising: a body configured to be worn on the skin and configured to couple to a transcutaneous analyte sensor, at least a portion of the body comprising a liquid crystal polymer.

Implementations of the embodiments may include one or more of the following. The body may comprise a housing. The body may be configured to retain one or more electrical components. The body may include a base and an enclosure configured to be coupled to the base such that a seal is formed between the base and the enclosure, and at least a portion of the enclosure comprises the liquid crystal polymer. The base may be configured to retain one or more electrical components within a perimeter of the base and the enclosure is configured to extend over the one or more electrical components. The enclosure may include an outer shell comprising the liquid crystal polymer. The outer shell may be positioned upon an inner shell of the enclosure comprising one or more of a nylon plastic, a polyolefin, or a thermoplastic elastomer. A filler may be disposed between the base and the enclosure. The filler may comprise a thermoplastic. The enclosure may be coupled to the base with an adhesive including polyolefin, polyurethane, silicone, epoxy, or acrylates. The seal between the base and the enclosure may be moisture proof. The seal between the base and the enclosure may be an ultrasonic weld, a laser weld, a vibration weld, or an electromagnetic weld. A patch may be configured to couple the body to the skin. The transcutaneous analyte sensor may be configured to extend from the body to be positioned within the skin. The body may be configured to retain one or more electrical components for receiving a signal from the transcutaneous analyte sensor.

In a second aspect, an on-skin wearable medical device configured to be deployed to a skin of a host, the on-skin wearable medical device comprising: a transcutaneous analyte sensor configured to generate a signal indicative of an analyte concentration in the host; and a body configured to be placed adjacent the skin of the host, the body including one or more bending sections configured to allow the body to bend to conform to a contour of the skin.

Implementations of the embodiments may include one or more of the following. The body may comprise a housing. The housing may have an exterior and an interior, and the one or more bending sections include one or more living hinges extending along the exterior of the housing. The exterior may include a top surface and a bottom surface, and the one or more living hinges extend along the top surface. The housing may be configured to bend along the one or more living hinges in a direction away from the bottom surface. The exterior may include a top surface and a bottom surface, and the one or more living hinges extend along the bottom surface. The housing may be configured to bend along the one or more living hinges in a direction away from the top surface. The one or more living hinges may comprise a channel extending along the exterior of the housing. The one or more living hinges may extend across the exterior along an axis central to the housing. The body may have an S-shape and each of the one or more bending sections includes a concavity forming the S-shape. The S-shape of the body may conform to an elliptical perimeter. The body may comprise a housing including a plurality of compartments, the plurality of compartments being separated by the one or more bending sections comprising a horizontal living hinge and a vertical living hinge. A flexible cover may be configured to extend over the housing and configured to bend with the housing. The one or more bending sections may comprise a cloth extending between rigid portions of the body. A flexible circuit board may be disposable in an interior of the body and configured to be coupled to one or more electrical components and bend with the body.

In a third aspect, an on-skin wearable medical device configured to be deployed to a skin of a host, the on-skin wearable medical device comprising: a transcutaneous analyte sensor configured to generate a signal indicative of an analyte concentration in the host; and a body configured to be placed adjacent the skin of the host, the body including one or more electrical conduits formed from an anisotropic conductive adhesive (ACA).

Implementations of the embodiments may include one or more of the following. The one or more electrical conduits may be configured to transmit electrical energy to or from the transcutaneous analyte sensor. The electrical energy may comprise an electrical signal to or from the transcutaneous analyte sensor. The electrical energy may comprise electrical power to or from the transcutaneous analyte sensor. The device may further comprise a battery, wherein the one or more electrical conduits transmit power from the battery to one or more electrical components positioned within the body. The one or more electrical components may comprise a wireless transmitter. The one or more electrical components may comprise one or more electrical terminals for the transcutaneous analyte sensor. The one or more electrical conduits may electrically connect one or more electrical components to a printed circuit board (PCB). The ACA may include particles having been collimated by an electric-field or a magnetic field being applied to the particles. The ACA may have been cured by heat or ultraviolet light.

In a fourth aspect, a method for forming one or more electrical conduits of an on-skin wearable medical device, the method comprising: positioning an anisotropic conductive adhesive (ACA) on an electrical substrate of the on-skin wearable medical device; collimating particles of the ACA; and curing the ACA to form the one or more electrical conduits.

Implementations of the embodiments may include one or more of the following. Conductivity of the one or more electrical conduits may be in a direction perpendicular to a plane of the electrical substrate. Collimating the particles of the ACA may include applying an electric-field or a magnetic field to the ACA. Curing the ACA may include applying heat or ultraviolet light to the ACA. The method may include forming the one or more electrical conduits between the electrical substrate and one or more electrical components of the on-skin wearable medical device. Curing the ACA couples the one or more electrical components to the electrical substrate physically and electrically. The one or more electrical conduits may be configured to transmit electrical energy to or from a transcutaneous analyte sensor. The electrical energy may comprise an electrical signal to or from the transcutaneous analyte sensor. The electrical energy may comprise electrical power to or from the transcutaneous analyte sensor. The one or more electrical conduits may be configured to transmit power from a battery to one or more electrical components positioned within a body of the on-skin wearable medical device.

In a fifth aspect, an on-skin wearable medical device configured to be deployed to a skin of a host, the on-skin wearable medical device comprising: a transcutaneous analyte sensor configured to generate a signal indicative of an analyte concentration in the host; and a body configured to be placed adjacent the skin of the host, a loop antenna coupled to the body and configured to receive signals or transmit signals from the body.

Implementations of the embodiments may include one or more of the following. The body may include a socket configured to couple to a plug coupled to the transcutaneous analyte sensor, the loop antenna surrounding the socket. The loop antenna may form at least a partial loop around the socket. The socket may include a gasket configured to form a seal with the plug. One or more electrical contacts may be positioned within the socket for contacting one or more electrical contacts coupled to the plug. One or more electrical contacts positioned within the socket may comprise an electrically conductive elastomeric material. The loop antenna may surround the one or more electrical contacts positioned within the socket. A channel may surround the socket, and the loop antenna is positioned within the channel. The loop antenna may have a rectangular cross section. The loop antenna may have an oblong shape.

In a sixth aspect, an on-skin wearable medical device configured to be deployed to a skin of a host, the on-skin wearable medical device comprising: a body including a sensor receiving portion having one or more slots; a transcutaneous analyte sensor configured to generate a signal indicative of an analyte concentration in the host, the transcutaneous analyte sensor positioned within the sensor receiving portion; and one or more conductor bodies each having a forked portion configured to couple to a portion of the transcutaneous analyte sensor, and each of the one or more conductor bodies configured to be pressed into a respective slot of the one or more slots to couple to the respective portion of the transcutaneous analyte sensor.

Implementations of the embodiments may include one or more of the following. Each of the one or more conductor bodies may include a lower surface and an upper surface, the lower surface including the forked portion and the upper surface including a releasable portion configured to release from an applicator for the one or more conductor bodies. The releasable portion may comprise a breakable portion of the one or more conductor bodies. The releasable portion may be configured to release from the applicator with a shearing force. The sensor receiving portion may include a channel extending transverse to the one or more slots, the transcutaneous analyte sensor extending along the channel. The one or more slots may each include a lower surface, and wherein the channel is raised above the respective lower surface. The one or more slots may each include a cavity extending from the channel to the respective lower surface, and the one or more one or more conductor bodies each include one or more arms configured to be positioned within a respective one of the cavities. The one or more conductor bodies may each be configured to be pressed into a respective slot of the one or more slots to apply pressure to the transcutaneous analyte sensor to retain the transcutaneous analyte sensor in place. The forked portion may have a wedge-shape and the one or more conductor bodies apply pressure to the transcutaneous analyte sensor from a vertical direction and from a lateral direction relative to the transcutaneous analyte sensor. The one or more conductor bodies may electrically couple the transcutaneous analyte sensor to one or more electrical components positioned within the body. The one or more slots may each include a lower surface and the forked portion is configured to face opposite the lower surface when the one or more conductor bodies are pressed into the respective slot of the one or more slots. The one or more conductor bodies may each have a vertex portion coupled to the forked portion, and the one or more slots each include a lower surface, and the vertex portion is configured to face towards the lower surface when the one or more conductor bodies are pressed into the respective slot of the one or more slots. The vertex portion may comprise a living hinge. The forked portion may include at least two arms having interior surfaces configured to face towards each other and extend parallel with each other when the one or more conductor bodies are pressed into the respective slot of the one or more slots. The interior surfaces may be configured to move towards each other upon the one or more conductor bodies being pressed into the respective slot of the one or more slots.

In a seventh aspect, a medical system comprising: an on-skin wearable medical device configured to be peeled onto a host's skin to at least partially deploy the on-skin wearable medical device to the host's skin.

Implementations of the embodiments may include one or more of the following. An applicator may be configured to at least partially deploy the on-skin wearable medical device to the host's skin. The applicator may include a reel configured to retain the on-skin wearable medical device. An elongate retainer body may be configured to retain the on-skin wearable medical device and wrap about the reel. The on-skin wearable medical device may be configured to release from the elongate retainer body to at least partially deploy to the host's skin. The elongate retainer body may comprise a ribbon. The elongate retainer body may be configured to be drawn from the reel. The elongate retainer body may be configured to retain a plurality of on-skin wearable medical devices. The elongate retainer body may be configured to be peeled along the host's skin and drawn from the applicator while being peeled along the host's skin. The on-skin wearable medical device may include a transcutaneous analyte sensor, and the applicator includes a needle configured to insert the transcutaneous analyte sensor into the host's skin. The applicator may be configured to retract the needle from the host's skin. The system may include a trigger assembly for inserting the needle into the host's skin. The trigger assembly may be actuated based on movement of the applicator relative to the host's skin. The applicator may include a first portion and a second portion, the first portion configured to retain a used needle following insertion into the host's skin and the second portion retaining a reel configured to retain the on-skin wearable medical device, the first portion being separable from the second portion. The applicator may include an insertion assembly configured to be positioned above the on-skin wearable medical device to insert a transcutaneous analyte sensor of the on-skin wearable medical device into the host's skin. At least a portion of the on-skin wearable medical device may be flexible. A flexible housing of the on-skin wearable medical device may be configured to be peeled onto the host's skin. The on-skin wearable medical device may be configured to be peeled onto the host's skin to insert a transcutaneous analyte sensor of the on-skin wearable medical device into the host's skin. The on-skin wearable medical device may be configured to retain one or more electrical components for the on-skin wearable medical device. One or more electrical components may include one or more of a battery, a transmitter, or contacts for a transcutaneous analyte sensor.

In an eighth aspect, a medical device applicator system comprising: a reel configured to retain a plurality of flexible on-skin wearable medical devices to the reel, the on-skin wearable medical devices configured to be deployed from the reel to a host's skin.

Implementations of the embodiments may include one or more of the following. At least one of the plurality of flexible on-skin wearable medical devices is configured to be peeled onto the host's skin to at least partially deploy the on-skin wearable medical device to the host's skin. The system may include an elongate retainer body configured to retain the on-skin wearable medical device and wrap about the reel. The elongate retainer body may be configured to be drawn from the reel. The system may include an insertion assembly for inserting a needle into the host's skin to insert a transcutaneous analyte sensor of one of the on-skin wearable medical devices into the host's skin. The system may include a retraction assembly for retracting a needle from the host's skin following insertion of a transcutaneous analyte sensor of one of the on-skin wearable medical devices into the host's skin with the needle. The system may include an applicator housing configured to retain the reel and be gripped by a user. The applicator housing may include an opening configured for an elongate retainer body configured to retain the on-skin wearable medical device to be drawn from. The system may include the plurality of flexible on-skin wearable medical devices. Each of the plurality of flexible on-skin wearable medical devices may include a transcutaneous analyte sensor configured to be inserted into the host's skin.

In a ninth aspect, an on-skin wearable medical device configured to be deployed to a skin of a host, the on-skin wearable medical device comprising: a body including a sensor receiving portion; a transcutaneous analyte sensor positioned within the sensor receiving portion; an electrical connector body including a first end portion, a second end portion, and a central portion between the first end portion and the second end portion, the first end portion being conductive and configured to form an electrical connection with a first portion of the transcutaneous analyte sensor, the second end portion being conductive and configured to form an electrical connection with a second portion of the transcutaneous analyte sensor, and the central portion comprising an insulator electrically insulating the first end portion from the second end portion; and a top member configured to be disposed on the transcutaneous analyte sensor and the electrical connector body.

Implementations of the embodiments may include one or more of the following. The first end portion may comprise an electrically conductive elastomeric material. The second end portion may comprise an electrically conductive elastomeric material. The central portion may comprise an elastomeric material. The electrical connector body may comprise a continuous body extending from the first end portion to the second end portion. The first end portion may be electrically connected with an electrical substrate, and the second end portion is electrically connected with an electrical substrate. The device may include at least one conductive tab configured to electrically connect the first end portion to an electrical substrate, and at least one conductive tab configured to electrically connect the second end portion to an electrical substrate. The sensor receiving portion may include a cavity configured to receive the transcutaneous analyte sensor, and the top member seals the cavity. The device may include an adhesive material configured to create a seal between the top member and the body. The first end portion and the second end portion may each be configured to electrically connect the transcutaneous analyte sensor with one or more electrical components of the body.

In a tenth aspect, an on-skin wearable medical device configured to be deployed to a skin of a host, the on-skin wearable medical device comprising: a housing configured to be worn on the skin and configured to couple to a transcutaneous analyte sensor, the housing including a wall comprising a film layer.

Implementations of the embodiments may include one or more of the following. The housing may include a bottom portion configured to be positioned proximate the skin and a top portion configured to be raised above the bottom portion, at least a portion of the top portion comprising the film layer. The housing may include an outer top surface configured to face away from the skin, at least a portion of the outer top surface comprising the film layer. The housing may include a bottom portion configured to be positioned proximate the skin, at least a portion of the bottom portion comprising the film layer. The housing may include a bottom surface configured to face towards the skin, at least a portion of the bottom surface comprising the film layer. The housing may include a bottom portion configured to be positioned proximate the skin and a top portion configured to be raised above the bottom portion, wherein the bottom portion includes the film layer and the top portion includes the film layer, the film layer of the bottom portion being coupled to the film layer of the top portion. The film layer of the bottom portion may couple to the film layer of the top portion to form a seal of an interior cavity of the housing. The film layer of the bottom portion may couple to the film layer of the top portion about a periphery of the housing. The film layer of the bottom portion may couple to a socket for receiving a plug coupled to the transcutaneous analyte sensor, and the film layer of the top portion couples to the socket for receiving the plug coupled to the transcutaneous analyte sensor. The device may include a patch coupled to the housing and configured to couple the housing to the skin. The housing may be flexible. The housing may have a length, a width, and a height, with the length and the width each being greater than the height. The device may include a socket for receiving a plug coupled to the transcutaneous analyte sensor. The socket may comprise an opening on a top portion of the housing. The length may be greater than the width and the socket has an oblong shape with a long dimension extending along the width of the housing. The housing may have a rectangular shape. The device may include a cover layer forming an outer surface of the housing and positioned over the film layer. The device may include one or more electrical components positioned within an interior cavity of the housing. The one or more electrical components may comprise one or more of a transmitter, a battery, or contacts for the transcutaneous analyte sensor. A filler may be positioned within an interior cavity of the housing.

In an eleventh aspect, an on-skin wearable medical device configured to be deployed to a skin of a host, the on-skin wearable medical device comprising: a body configured to be worn on the skin; and a socket coupled to the body and configured to couple to a plug coupled to a transcutaneous analyte sensor, the socket including a raised portion and a channel surrounding the raised portion, the raised portion including one or more electrical contacts for electrical connection with the transcutaneous analyte sensor and the channel including a fluid disposed therein for forming a seal with at least a portion of the plug.

Implementations of the embodiments may include one or more of the following. The fluid may comprise a gel. The gel may comprise petroleum jelly. The channel may include a reservoir for receiving and storing any excess amount of the fluid following the coupling of the plug to the socket. A raised outer wall may surround the channel. The raised outer wall may contour to a shape of the plug. The raised portion may include a coupler for coupling with the plug. The raised portion may be configured to enter a cavity of the plug. The raised portion may include a top surface that the one or more electrical contacts are positioned on, the top surface configured to face the plug, and the socket includes a bottom surface facing opposite the top surface, the one or more electrical contacts passing from the top surface to the bottom surface. The one or more electrical contacts may comprise an electrically conductive elastomeric material.

In a twelfth aspect, an on-skin wearable medical device configured to be deployed to a skin of a host, the on-skin wearable medical device comprising: an elongate housing configured to be worn on the skin and having a long dimension; and an elongate socket coupled to the elongate housing and configured to couple to a plug coupled to a transcutaneous analyte sensor, the elongate socket having a long dimension extending along the long dimension of the elongate housing.

Implementations of the embodiments may include one or more of the following. The elongate socket may comprise an upper level positioned above a lower level of the elongate housing. The lower level may include one or more electrical components comprising one or more of a transmitter or a battery. The elongate socket may be positioned above a transmitter. The elongate housing may comprise a co-molded material. The co-molded material may include a first material having a greater stiffness than a second material. The first material may comprise a frame that the second material is coupled to, and the second material forms at least a portion of an outer surface of the elongate housing. The elongate housing may be flexible. The elongate housing may include a bottom portion configured to be positioned proximate the skin and a top portion configured to be raised above the bottom portion, wherein the bottom portion includes a film layer. A patch may be coupled to the bottom portion, the patch configured to couple the elongate housing to the skin.

In a thirteenth aspect, an on-skin wearable medical device configured to be deployed to a skin of a host, the on-skin wearable medical device comprising: a body configured to be worn on the skin and configured to couple to a transcutaneous analyte sensor; one or more electrical components positioned within the body; at least one power source positioned within the body and configured to provide power to the one or more electrical components; and a conductive tape configured to electrically couple the at least one power source to the one or more electrical components.

Implementations of the embodiments may include one or more of the following. The at least one power source may comprise at least one battery. The conductive tape may be configured to couple to a positive terminal or a negative terminal of the at least one battery. The conductive tape may be flexible. The conductive tape may wrap around at least a portion of the at least one power source.

In a fourteenth aspect, a system comprising: a first conductive film; a second conductive film configured to be disposed next to the first conductive film; a non-conductive film configured to be laterally sandwiched by the first conductive film and the second conductive film; a transcutaneous analyte sensor disposed over the first conductive film, the second conductive film, and the non-conductive film, and in electrical contact with the first conductive film and the second conductive film; and a barrier film configured to be disposed over the transcutaneous analyte sensor to create a seal over the transcutaneous analyte sensor, the first conductive film, the second conductive film, and the non-conductive film, and wherein the first conductive film, the second conductive film, and the non-conductive film are further configured to couple to an electrical substrate to form an electrical connection between the transcutaneous analyte sensor and the electrical substrate.

Implementations of the embodiments may include one or more of the following. A hardened backing layer may be configured to be disposed over the barrier film. The transcutaneous analyte sensor may be further configured to bend at a region away from the first conductive film, the second conductive film, and the non-conductive film. The barrier film may be non-conductive. The transcutaneous analyte sensor may have a tip configured to be disposed over the first conductive film, and the first conductive film is a working electrode, and the second conductive film is a reference electrode.

In a fifteenth aspect, an on-skin wearable medical device configured to be deployed to a skin of a host, the on-skin wearable medical device comprising: a body configured to be worn on the skin and configured to couple to a transcutaneous analyte sensor; an electrical substrate positioned within the body; at least one battery having a perimeter and an electrical terminal, the electrical terminal being positioned upon the electrical substrate, the at least one battery configured to provide power to one or more electrical components of the on-skin wearable medical device; and a seal positioned at the perimeter of the at least one battery and configured to reduce moisture ingress to the electrical terminal.

Implementations of the embodiments may include one or more of the following. The electrical substrate may include an electrical contact, and the electrical terminal contacts the electrical contact, and the seal is configured to reduce moisture ingress to the contact between the electrical terminal and the electrical contact. The seal may be positioned upon the electrical substrate. The seal may extend about an entirety of the perimeter of the at least one battery. The battery may comprise a coin-cell battery. The electrical terminal of the coin-cell battery may be a negative terminal. The perimeter may be circular. The body may include a housing having an interior cavity, and the seal comprises a curable material filling the interior cavity. The at least one battery may have a lower surface and an upper surface, the lower surface of the at least one battery facing the electrical substrate, and the seal covering the upper surface of the at least one battery. The seal may comprise one or more of a curable material or a foam.

In a sixteenth aspect, an on-skin wearable medical device configured to be deployed to a skin of a host, the on-skin wearable medical device comprising: a housing configured to be worn on the skin and configured to couple to a transcutaneous analyte sensor, the housing including one or more walls defining a cavity for receiving a curable material, the cavity having an end portion with an opening; a substrate positioned within the housing and at the opening of the cavity; curable material disposed within the cavity; and an adhesive layer positioned between the substrate and the opening of the cavity, the adhesive layer configured to impede the curable material from egressing the cavity through the opening.

Implementations of the embodiments may include one or more of the following. The adhesive layer may be positioned upon the substrate. The substrate may comprise an electrical substrate configured to electrically couple with the transcutaneous analyte sensor. The one or more walls may each have ends defining the opening of the cavity, and the adhesive layer contacts the ends of the one or more walls and the substrate. The adhesive layer may include a first surface and a second surface facing opposite the first surface, and the first surface and the second surface each include an adhesive. The first surface may contact the substrate. The cavity may be configured to receive the transcutaneous analyte sensor. The adhesive layer may be configured to electrically couple to the transcutaneous analyte sensor. The adhesive layer may be configured to impede the curable material from flowing through the opening onto a portion of the substrate that is positioned outward of the opening.

In a seventeenth aspect, an on-skin wearable medical device configured to be deployed to a skin of a host, the on-skin wearable medical device comprising: a body configured to be worn on the skin and configured to couple to a transcutaneous analyte sensor, at least a portion of the body comprising a hotmelt material.

Implementations of the embodiments may include one or more of the following. The body may comprise a housing. The body may be configured to retain one or more electrical components. The body may include a base, an enclosure, and a filler disposed between the base and the enclosure. The filler may comprise the hotmelt material. The base and the enclosure may be composed of a first material, wherein the hotmelt material is configured to be molded at a lower pressure or temperature than the first material. The hotmelt material may be configured to set more quickly than materials that the base and the enclosure are at least partially composed of following molding. The enclosure may be made of a material having a greater hardness or reduced moisture permeability than the hotmelt material. The base may be composed of a material having a greater hardness, cohesiveness, or abrasion resistance than the hotmelt material. The base may be composed of a material having a reduced moisture permeability than the hotmelt material. The enclosure and the base may be molded from one or more of poly(methyl methacrylate) (PMMA), acrylonitrile butadiene styrene (ABS), nylon (polyamide, PA), polycarbonate (PC), polyethylene (PE), polyoxymethylene (POM), polypropylene (PP), polystyrene (PS), thermoplastic elastomer (TPE), or thermoplastic polyurethane (TPU). The base may be composed of a different material than the enclosure. The base and the enclosure may be composed of a same material. The filler may be composed of a different material than the base and the enclosure. The base may be composed of a first polymer, and the enclosure is composed of a second polymer that is different than the first polymer. An electrical substrate may be positioned between the base and the enclosure. The filler may be configured to surround the electrical substrate. The filler may occupy a void between the base and the enclosure. The enclosure may be configured to be coupled to the base such that a seal is formed between the base and the enclosure. The hotmelt material may be configured to be molded by low pressure molding. The hotmelt material may be configured to set more quickly than a thermoset polymer following molding. The body may include a first housing, a second housing, and a filler between the first housing and the second housing. A patch may be configured to couple the body to the skin. The transcutaneous analyte sensor may be configured to extend from the body to be positioned within the skin. The body may be configured to retain one or more electrical components for receiving a signal from the transcutaneous analyte sensor.

In further aspects and embodiments, the above methods and features of the various aspects are formulated in terms of a system as in various aspects, having an applicator configured to carry out the method features. Any of the features of an embodiment of any of the aspects, including but not limited to any embodiments of any of the first through seventeenth aspects referred to above, is applicable to all other aspects and embodiments identified herein, including but not limited to any embodiments of any of the first through seventeenth aspects referred to above. Moreover, any of the features of an embodiment of the various aspects, including but not limited to any embodiments of any of the first through seventeenth aspects referred to above, is independently combinable, partly or wholly with other embodiments described herein in any way, e.g., one, two, or three or more embodiments may be combinable in whole or in part. Further, any of the features of an embodiment of the various aspects, including but not limited to any embodiments of any of the first through seventeenth aspects referred to above, may be made optional to other aspects or embodiments. Any aspect or embodiment of a method can be performed by a system or apparatus of another aspect or embodiment, and any aspect or embodiment of a system or apparatus can be configured to perform a method of another aspect or embodiment, including but not limited to any embodiments of any of the first through seventeenth aspects referred to above.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar embodiments.

FIG. 1 is a schematic view of an analyte sensor system attached to a host and communicating with a plurality of example devices.

FIG. 2 is a block diagram that illustrates electronics associated with the analyte sensor system of FIG. 1.

FIGS. 3A-3C illustrate an on-skin wearable medical device having a transcutaneous analyte sensor.

FIG. 4A illustrates a perspective view of an on-skin wearable medical device.

FIG. 4B illustrates a top view of the on-skin wearable medical device shown in FIG. 4A.

FIG. 4C illustrates an exploded view of the on-skin wearable medical device of FIG. 4A.

FIG. 4D illustrates a cross-sectional view of a section of the on-skin wearable medical device of FIG. 4A.

FIG. 4E illustrates a cross-sectional exploded view of a section of the on-skin wearable medical device of FIG. 4A.

FIG. 4F illustrates a cross-sectional view of a section of the on-skin wearable medical device shown in FIG. 4E.

FIG. 5A illustrates a top perspective view of a socket and a plug of an on-skin wearable medical device with the plug detached from the socket.

FIG. 5B illustrates a bottom perspective view of the socket and the plug of the on-skin wearable medical device shown in FIG. 5A with the plug detached from the socket.

FIG. 5C illustrates an isolated bottom perspective view of the plug of FIG. 5A.

FIG. 5D illustrates a magnified cross-sectional view of the plug of FIG. 5A attached to the socket of FIG. 5A.

FIG. 6A illustrates a top perspective view of an on-skin wearable medical device.

FIG. 6B illustrates a side perspective view of the on-skin wearable medical device of FIG. 6A.

FIG. 6C illustrates a top perspective view of the on-skin wearable medical device of FIG. 6A with a detached plug.

FIG. 6D illustrates a bottom perspective view of the plug of FIG. 6C.

FIG. 6E illustrates a top perspective exploded view of the on-skin wearable medical device of FIG. 6A.

FIG. 6F illustrates a bottom perspective exploded view of the on-skin wearable medical device of FIG. 6A.

FIG. 6G illustrates an isolated perspective view of a first portion of a housing of the on-skin wearable medical device.

FIG. 6H illustrates an isolated bottom perspective view of the first portion of the housing of the on-skin wearable medical device of FIG. 6G.

FIG. 6I illustrates an isolated perspective view of the housing of the on-skin wearable medical device of FIG. 6A.

FIG. 7 illustrates a perspective view of a housing of an on-skin wearable medical device.

FIG. 8A illustrates a top perspective view of a socket of an on-skin wearable medical device.

FIG. 8B illustrates a bottom perspective view of the socket of FIG. 8A.

FIG. 8C illustrates a side view of the socket of FIG. 8A.

FIG. 8D illustrates a top view of the socket of FIG. 8A.

FIG. 8E illustrates a cross-sectional view of the socket of FIG. 8A along line A-A shown in FIG. 8A.

FIG. 8F illustrates an isolated perspective view of a loop antenna of FIG. 8A.

FIG. 9A illustrates a perspective view of components of a sensor connection of an on-skin wearable medical device in a pre-assembled state.

FIG. 9B illustrates a perspective view of the components of the sensor connection of FIG. 9A with a sensor positioned within a channel.

FIG. 9C illustrates a perspective view of the components of the sensor connection of FIG. 9A with a plurality of conductors coupled to the sensor.

FIG. 9D illustrates a perspective view of the components of the sensor connection of FIG. 9A in an assembled state.

FIG. 10A illustrates an exploded view of components of a sensor connection.

FIG. 10B illustrates a cross-sectional perspective view of the sensor connection of FIG. 10A.

FIG. 10C illustrates a top view of the sensor connection of FIG. 10A.

FIG. 11A illustrates a perspective view of components of a sensor connection.

FIG. 11B illustrates an exploded cross-sectional view of components of the sensor connection shown in FIG. 11A.

FIG. 11C illustrates a perspective cross-sectional view of components of a sensor connection.

FIG. 11D illustrates a top view of the sensor connection of FIG. 11A.

FIG. 12A illustrates a cross-sectional view of an anisotropic conductive adhesive (ACA) within a body of an on-skin wearable medical device.

FIG. 12B illustrates a cross-sectional view of the ACA of FIG. 12A formed into electrical conduits.

FIG. 12C illustrates a cross-sectional view of the ACA of FIG. 12A cured.

FIG. 12D is a flow chart of a method.

FIG. 13A illustrates a perspective view of an on-skin wearable medical device.

FIG. 13B illustrates an exploded view of an on-skin wearable medical device.

FIG. 14 illustrates an exploded view of an on-skin wearable medical device.

FIG. 15 illustrates an exploded view of an on-skin wearable medical device.

FIG. 16A illustrates a top perspective view of a system configured to deploy an on-skin wearable medical device, with components shown in transparency.

FIG. 16B illustrates a bottom perspective view of a system configured to deploy an on-skin wearable medical device, with components shown in transparency.

FIG. 16C illustrates a side schematic view of the system shown in FIG. 16A being applied to skin.

FIG. 16D illustrates a side schematic view of the system shown in FIG. 16A being peeled onto a host's skin.

FIG. 16E illustrates a side schematic view of the system shown in FIG. 16A inserting a needle into the host's skin.

FIG. 16F illustrates a side schematic view of the system shown in FIG. 16A having inserted a transcutaneous analyte sensor into the host's skin.

FIG. 16G illustrates a side schematic view of the system shown in FIG. 16A removed from the host's skin, with a first portion of the applicator separated from a second portion of the applicator.

FIG. 17A illustrates a top perspective view of an on-skin wearable medical device.

FIG. 17B illustrates a top perspective view of the on-skin wearable medical device of FIG. 17A in a bent state.

FIG. 17C illustrates a bottom perspective view of the on-skin wearable medical device of FIG. 17A.

FIG. 17D illustrates a side view of the on-skin wearable medical device of FIG. 17A.

FIG. 17E illustrates a top view of the on-skin wearable medical device of FIG. 17A, with components shown in transparency.

FIG. 18A illustrates a top perspective view of an on-skin wearable medical device.

FIG. 18B illustrates a top perspective view of the on-skin wearable medical device of FIG. 18A in a bent state.

FIG. 18C illustrates a bottom perspective view of the on-skin wearable medical device of FIG. 18A.

FIG. 18D illustrates a side view of the on-skin wearable medical device of FIG. 18A.

FIG. 18E illustrates a top view of the on-skin wearable medical device of FIG. 18A with components shown in transparency.

FIG. 19A illustrates a top perspective view of an on-skin wearable medical device.

FIG. 19B illustrates a top perspective view of the on-skin wearable medical device of FIG. 19A in a bent state.

FIG. 19C illustrates a bottom perspective view of the on-skin wearable medical device of FIG. 19A.

FIG. 19D illustrates a side view of the on-skin wearable medical device of FIG. 19A.

FIG. 19E illustrates a top view of the on-skin wearable medical device of FIG. 19A, with components shown in transparency.

FIG. 20A illustrates a top view of an on-skin wearable medical device.

FIG. 20B illustrates a perspective view of the on-skin wearable medical device of FIG. 20A.

FIG. 21A illustrates a perspective view of an on-skin wearable medical device.

FIG. 21B illustrates an exploded view of the on-skin wearable medical device of FIG. 21A.

FIG. 22A illustrates a perspective view of an on-skin wearable medical device.

FIG. 22B illustrates a cross-sectional view of the on-skin wearable medical device of FIG. 22A.

FIG. 23A illustrates a top view of an on-skin wearable medical device.

FIG. 23B illustrates a perspective view of the on-skin wearable medical device of FIG. 23A.

FIG. 24 illustrates a perspective view of an on-skin wearable medical device.

FIG. 25A illustrates a top view of an on-skin wearable medical device.

FIG. 25B illustrates a perspective view of the on-skin wearable medical device of FIG. 25A.

FIG. 26 illustrates a perspective view of components of a sensor connection.

FIG. 27 illustrates a perspective view of a sensor connection being assembled.

FIG. 28 illustrates a cross-sectional view of a sensor connection.

FIG. 29 illustrates a detail view of a sensor connection.

FIG. 30A illustrates a sensor assembly being inserted into a housing.

FIG. 30B illustrates a sensor assembly inserted into a housing.

FIG. 31 illustrates a cross-sectional view of a conductor body and an analyte sensor.

FIG. 32A illustrates a perspective view of an analyte sensor being inserted into a sensor receiving portion.

FIG. 32B illustrates a perspective view of an analyte sensor inserted into a sensor receiving portion.

FIG. 33 illustrates a cross-sectional view of a conductor body inserted into a sensor receiving portion.

FIG. 34 illustrates a cross-sectional view of a battery upon a substrate.

FIG. 35 illustrates a top view of the battery of FIG. 34 upon a substrate.

FIG. 36 illustrates a cross-sectional view of a battery upon a substrate.

FIG. 37 illustrates a cross-sectional view of a curable material egressing a cavity.

FIG. 38 illustrates a cross-sectional view of a curable material retained within a cavity.

### DETAILED DESCRIPTION

The following description and examples illustrate some example implementations of the disclosure in detail. Those of skill in the art will recognize that there are numerous variations and modifications of the disclosure that are encompassed by its scope. Accordingly, the description of a certain example implementation should not be deemed to limit the scope of the present disclosure.

In vivo analyte sensing technology may rely on in vivo sensors. In vivo sensors may include an elongated conductive body having one or more electrodes such as a working electrode and a reference electrode.

For example, a platinum metal-clad, tantalum wire is sometimes used as a core bare sensing element with one or more reference or counter electrodes for an analyte sensor. This sensing element is coated in membranes to yield the final sensor. Other forms of sensors that may be utilized are disclosed in U.S. patent application Ser. No. 16/854,647, entitled “Preconnected Analyte Sensors,” and filed on Apr. 21, 202, published U.S. Publication No. 2020/0330010 on Oct. 22, 2020, herein incorporated by reference in its entirety.

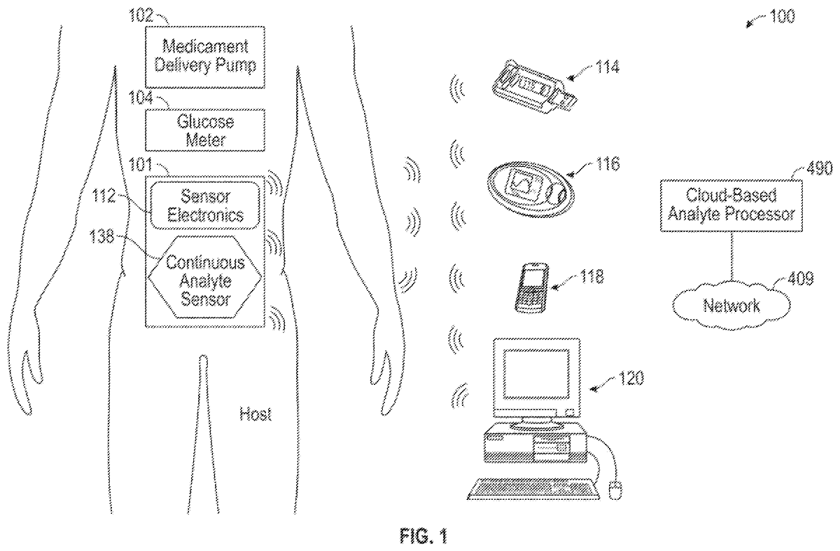

FIG. 1 depicts an example system 100, in accordance with some example implementations. The system 100 may include an analyte sensor system 101 including sensor electronics 112 and an analyte sensor 138. The system 100 may include other medical devices and/or sensors, such as medicament delivery pump 102 and glucose meter 104. The analyte sensor 138 may be physically connected to sensor electronics 112 and may be integral with (e.g., non-releasably attached to) or releasably attachable to the sensor electronics. For example, in some implementations, continuous analyte sensor 138 may be connected to sensor electronics 112 via a sensor carrier or sensor connection that mechanically and electrically interfaces the analyte sensor 138 with the sensor electronics. In some other implementations, continuous analyte sensor 138 may be directly connected to sensor electronics 112 without utilization of a sensor carrier that mechanically and electrically interfaces the analyte sensor 138 with the sensor electronics. The sensor electronics 112, medicament delivery pump 102, and/or glucose meter 104 may couple with one or more devices, such as display devices 114, 116, 118, and/or 120.

In some example implementations, the system 100 may include a cloud-based analyte processor 490 configured to analyze analyte data (and/or other patient-related data) provided via network 409 (e.g., via wired, wireless, or a combination thereof) from sensor system 101 and other devices, such as display devices 114, 116, 118, and/or 120 and the like, associated with the host (also referred to as a patient) and generate reports providing high-level information, such as statistics, regarding the measured analyte over a certain time frame. A full discussion of using a cloud-based analyte processing system may be found in U.S. patent application Ser. No. 13/788,375, entitled “Cloud-Based Processing of Analyte Data” and filed on Mar. 7, 2013, published as U.S. Patent Application Publication 2013/0325352, herein incorporated by reference in its entirety. In some implementations, one or more steps of the factory calibration algorithm can be performed in the cloud.

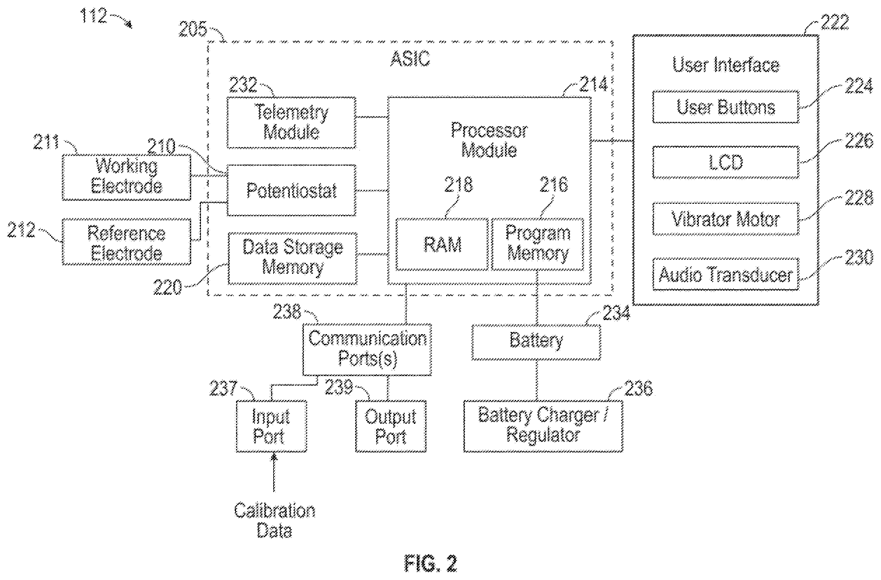

In some example implementations, electrical components in the form of sensor electronics 112 may include electronic circuitry associated with measuring and processing data generated by the analyte sensor 138. This generated analyte sensor data may also include algorithms, which can be used to process and calibrate the analyte sensor data, although these algorithms may be provided in other ways as well. The sensor electronics 112 may include hardware, firmware, software, or a combination thereof, to provide measurement of levels of the analyte via an analyte sensor, such as a glucose sensor. An example implementation of the sensor electronics 112 is described further below with respect to FIG. 2. In one implementation, the factory calibration algorithms described herein may be performed by the sensor electronics.

The sensor electronics 112 may, as noted, couple (e.g., wirelessly and the like) with one or more devices, such as display devices 114, 116, 118, and/or 120. The display devices 114, 116, 118, and/or 120 may be configured for presenting information (and/or alarming), such as sensor information transmitted by the sensor electronics 112 for display at the display devices 114, 116,118, and/or 120. In one implementation, the factory calibration algorithms described herein may be performed at least in part by the display devices.

In some example implementations, the relatively small, key fob-like display device 114 may comprise a wrist watch, a belt, a necklace, a pendent, a piece of jewelry, an adhesive patch, a pager, a key fob, a plastic card (e.g., credit card), an identification (ID) card, and/or the like. This small display device 114 may include a relatively small display (e.g., smaller than the large display device 116) and may be configured to display certain types of displayable sensor information, such as a numerical value, and an arrow, or a color code.

In some example implementations, the relatively large, hand-held display device 116 may comprise a hand-held receiver device, a palm-top computer, and/or the like. This large display device may include a relatively larger display (e.g., larger than the small display device 114) and may be configured to display information, such as a graphical representation of the sensor data including current and historic sensor data output by sensor system 100.

In some example implementations, the analyte sensor 138 may comprise a transcutaneous analyte sensor. The transcutaneous analyte sensor may be configured to generate a signal indicative of an analyte concentration in a host. The analyte sensor 138 may comprise a glucose sensor configured to measure glucose in the blood or interstitial fluid using one or more measurement techniques, such as enzymatic, chemical, physical, electrochemical, spectrophotometric, polarimetric, calorimetric, iontophoretic, radiometric, immunochemical, and the like. In implementations in which the analyte sensor 138 includes a glucose sensor, the glucose sensor may comprise any device capable of measuring the concentration of glucose and may use a variety of techniques to measure glucose including invasive, minimally invasive, and non-invasive sensing techniques (e.g., fluorescence monitoring), to provide data, such as a data stream, indicative of the concentration of glucose in a host. The data stream may be sensor data (raw and/or filtered), which may be converted into a calibrated data stream used to provide a value of glucose to a host, such as a user, a patient, or a caretaker (e.g., a parent, a relative, a guardian, a teacher, a doctor, a nurse, or any other individual that has an interest in the wellbeing of the host). Moreover, the analyte sensor 138 may be implanted as at least one of the following types of analyte sensors: an implantable glucose sensor, a transcutaneous glucose sensor, implanted in a host vessel or extracorporeally, a subcutaneous sensor, a refillable subcutaneous sensor, an intravascular sensor.

Although the disclosure herein refers to some implementations that include an analyte sensor 138 comprising a glucose sensor, the analyte sensor 138 may comprise other types of analyte sensors as well. Moreover, although some implementations refer to the glucose sensor as an implantable glucose sensor, other types of devices capable of detecting a concentration of glucose and providing an output signal representative of glucose concentration may be used as well. Furthermore, although the description herein refers to glucose as the analyte being measured, processed, and the like, other analytes may be used as well including, for example, ketone bodies (e.g., acetone, acetoacetic acid and beta hydroxybutyric acid, lactate, etc.), glucagon, acetyl-CoA, triglycerides, fatty acids, intermediaries in the citric acid cycle, choline, insulin, cortisol, testosterone, and the like.

In some manufacturing systems, sensors 138 are manually sorted, placed and held in fixtures. These fixtures are manually moved from station to station during manufacturing for various process steps including interfacing electrical measurement equipment for testing and calibration operations. However, manual handling of sensors can be inefficient, can cause delays due to non-ideal mechanical and electrical connections, and can risk damage to the sensor and/or testing and calibration equipment and can induce sensor variability that can lead to inaccurate verification data being collected in manufacturing. In addition, the process of packaging sensor 138 with the sensor electronics 112 into a wearable device involves further manual manipulation of the sensor that can damage sensor 138.

Identification and other data associated with each sensor may be stored on the sensor carrier, if utilized, for logging and tracking of each sensor during manufacturing, testing, calibration, and in vivo operations. Following testing and calibration operations, the sensor carrier may be used to connect the sensor to sensor electronics of a wearable device, such as an on-skin sensor assembly, in an arrangement that is sealed and electrically robust. In an implementation not incorporating such a sensor carrier, the sensor may be directly connected to the sensor electronics (e.g. to the printed circuit board of the sensor electronics) of the wearable device.

FIG. 2 depicts an example of electronics 112 that may be used in sensor electronics 112 or may be implemented in a manufacturing station such as a testing station, a calibration station, a smart carrier, or other equipment used during manufacturing of device 101, in accordance with some example implementations. The sensor electronics 112 may include electronics components that are configured to process sensor information, such as sensor data, and generate transformed sensor data and displayable sensor information, e.g., via a processor module. For example, the processor module may transform sensor data into one or more of the following: filtered sensor data (e.g., one or more filtered analyte concentration values), raw sensor data, calibrated sensor data (e.g., one or more calibrated analyte concentration values), rate of change information, trend information, rate of acceleration/deceleration information, sensor diagnostic information, location information, alarm/alert information, calibration information such as may be determined by factory calibration algorithms as disclosed herein, smoothing and/or filtering algorithms of sensor data, and/or the like.

In some implementations, a processor module 214 is configured to achieve a substantial portion, if not all, of the data processing, including data processing pertaining to factory calibration. Processor module 214 may be integral to sensor electronics 112 and/or may be located remotely, such as in one or more of devices 114, 116, 118, and/or 120 and/or cloud 490. For example, in some implementations, processor module 214 may be located at least partially within a cloud-based analyte processor 490 or elsewhere in network 409.

In some example implementations, the processor module 214 may be configured to calibrate the sensor data, and the data storage memory 220 may store the calibrated sensor data points as transformed sensor data. Moreover, the processor module 214 may be configured, in some example implementations, to wirelessly receive calibration information from a display device, such as devices 114, 116, 118, and/or 120, to enable calibration of the sensor data from sensor 138. Furthermore, the processor module 214 may be configured to perform additional algorithmic processing on the sensor data (e.g., calibrated and/or filtered data and/or other sensor information), and the data storage memory 220 may be configured to store the transformed sensor data and/or sensor diagnostic information associated with the algorithms. The processor module 214 may further be configured to store and use calibration information determined from a factory calibration, as described below.

In some example implementations, the sensor electronics 112 may comprise an application-specific integrated circuit (ASIC) 205 coupled to a user interface 222. The ASIC 205 may further include a potentiostat 210, a telemetry module 232 for transmitting data from the sensor electronics 112 to one or more devices, such as devices 114, 116, 118, and/or 120, and/or other components for signal processing and data storage (e.g., processor module 214 and data storage memory 220). Although FIG. 2 depicts ASIC 205, other types of circuitry may be used as well, including field programmable gate arrays (FPGA), one or more microprocessors configured to provide some (if not all of) the processing performed by the sensor electronics 112, analog circuitry, digital circuitry, or a combination thereof.

In the example depicted in FIG. 2, through a first input port for sensor data the potentiostat 210 is coupled to an analyte sensor 138, such as a glucose sensor to generate sensor data from the analyte. The potentiostat 210 may be coupled to a working electrode 211 and reference electrode 212 that form a part of sensor 138. The potentiostat may provide a voltage to one of the electrodes 211, 212 of analyte sensor 138 to bias the sensor for measurement of a value (e.g., a current) indicative of the analyte concentration in a host (also referred to as the analog portion of the sensor). The potentiostat 210 may have one or more connections to sensor 138 depending on the number of electrodes incorporated into the analyte sensor 138 (such as a counter electrode as a third electrode).

In some example implementations, the potentiostat 210 may include a resistor that translates a current value from sensor 138 into a voltage value, while in some example implementations, a current-to-frequency converter (not shown) may also be configured to integrate continuously a measured current value from sensor 138 using, for example, a charge-counting device. In some example implementations, an analog-to-digital converter (not shown) may digitize the analog signal from sensor 138 into so-called “counts” to allow processing by the processor module 214. The resulting counts may be directly related to the current measured by the potentiostat 210, which may be directly related to an analyte level, such as a glucose level, in the host.

The telemetry module 232 may be operably connected to processor module 214 and may provide the hardware, firmware, and/or software that enable wireless communication between the sensor electronics 112 and one or more other devices, such as display devices, processors, network access devices, and the like. A variety of wireless radio technologies that can be implemented in the telemetry module 232 include Bluetooth, Bluetooth Low-Energy, ANT, ANT+, ZigBee, IEEE 802.11, IEEE 802.16, cellular radio access technologies, radio frequency (RF), infrared (IR), paging network communication, magnetic induction, satellite data communication, spread spectrum communication, frequency hopping communication, near field communications, and/or the like. In some example implementations, the telemetry module 232 comprises a Bluetooth chip, although Bluetooth technology may also be implemented in a combination of the telemetry module 232 and the processor module 214. The telemetry module 232 may comprise a transmitter in implementations.

The processor module 214 may control the processing performed by the sensor electronics 112. For example, the processor module 214 may be configured to process data (e.g., counts), from the sensor, filter the data, calibrate the data, perform fail-safe checking, and/or the like.

Potentiostat 210 may measure the analyte (e.g., glucose and/or the like) at discrete time intervals or continuously, for example, using a current-to-voltage or current-to-frequency converter.

The processor module 214 may further include a data generator (not shown) configured to generate data packages for transmission to devices, such as the display devices 114, 116, 118, and/or 120. Furthermore, the processor module 214 may generate data packets for transmission to these outside sources via telemetry module 232. In some example implementations, the data packages may include an identifier code for the sensor and/or sensor electronics 112, raw data, filtered data, calibrated data, rate of change information, trend information, error detection or correction, and/or the like.

The processor module 214 may also include a program memory 216 and other memory 218. The processor module 214 may be coupled to a communications interface, such as a communication port 238, and a power source, such as a battery 234. Moreover, the battery 234 may be further coupled to a battery charger and/or regulator 236 to provide power to sensor electronics 112 and/or charge the battery 234.

The program memory 216 may be implemented as a semi-static memory for storing data, such as an identifier for a coupled sensor 138 (e.g., a sensor identifier (ID)) and for storing code (also referred to as program code) to configure the ASIC 205 to perform one or more of the operations/functions described herein. For example, the program code may configure processor module 214 to process data streams or counts, filter, perform the calibration methods described below, perform fail-safe checking, and the like.

The memory 218 may also be used to store information. For example, the processor module 214 including memory 218 may be used as the system's cache memory, where temporary storage is provided for recent sensor data received from the sensor. In some example implementations, the memory may comprise memory storage components, such as read-only memory (ROM), random-access memory (RAM), dynamic-RAM, static-RAM, non-static RAM, electrically erasable programmable read only memory (EEPROM), rewritable ROMs, flash memory, and the like.

The data storage memory 220 may be coupled to the processor module 214 and may be configured to store a variety of sensor information. In some example implementations, the data storage memory 220 stores one or more days of analyte sensor data. The stored sensor information may include one or more of the following: a time stamp, raw sensor data (one or more raw analyte concentration values), calibrated data, filtered data, transformed sensor data, and/or any other displayable sensor information, calibration information (e.g., reference BG values and/or prior calibration information such as from factory calibration), sensor diagnostic information, and the like.

The user interface 222 may include a variety of interfaces, such as one or more buttons 224, a liquid crystal display (LCD) 226, a vibrator 228, an audio transducer (e.g., speaker) 230, a backlight (not shown), and/or the like. The components that comprise the user interface 222 may provide controls to interact with the user (e.g., the host).

The power source or battery 234 may be operatively connected to the processor module 214 (and possibly other components of the sensor electronics 112) and provide the necessary power for the sensor electronics 112. In other implementations, the receiver can be transcutaneously powered via an inductive coupling, for example.

A battery charger and/or regulator 236 may be configured to receive energy from an internal and/or external charger. In some example implementations, the battery 234 (or batteries) is configured to be charged via an inductive and/or wireless charging pad, although any other charging and/or power mechanism may be used as well.

One or more communication ports 238, also referred to as external connector(s), may be provided to allow communication with other devices, for example a PC communication (com) port can be provided to enable communication with systems that are separate from, or integral with, the sensor electronics 112. The communication port, for example, may comprise a serial (e.g., universal serial bus or “USB”) communication port, and allow for communicating with another computer system (e.g., PC, personal digital assistant or “PDA,” server, or the like). In some example implementations, factory information may be sent to the algorithm from the sensor or from a cloud data source.

The one or more communication ports 238 may further include an input port 237 in which calibration data may be received, and an output port 239 which may be employed to transmit calibrated data, or data to be calibrated, to a receiver or mobile device. FIG. 2 illustrates these aspects schematically. It will be understood that the ports may be separated physically, but in alternative implementations a single communication port may provide the functions of both the second input port and the output port.

In some analyte sensor systems, an on-skin portion of the sensor electronics may be simplified to minimize complexity and/or size of on-skin electronics, for example, providing only raw, calibrated, and/or filtered data to a display device configured to run calibration and other algorithms required for displaying the sensor data. However, the sensor electronics 112 (e.g., via processor module 214) may be implemented to execute prospective algorithms used to generate transformed sensor data and/or displayable sensor information, including, for example, algorithms that: evaluate a clinical acceptability of reference and/or sensor data, evaluate calibration data for best calibration based on inclusion criteria, evaluate a quality of the calibration, compare estimated analyte values with time corresponding measured analyte values, analyze a variation of estimated analyte values, evaluate a stability of the sensor and/or sensor data, detect signal artifacts (noise), replace signal artifacts, determine a rate of change and/or trend of the sensor data, perform dynamic and intelligent analyte value estimation, perform diagnostics on the sensor and/or sensor data, set modes of operation, evaluate the data for aberrancies, and/or the like. The sensor electronics 112 may comprise a transmitter in implementations.

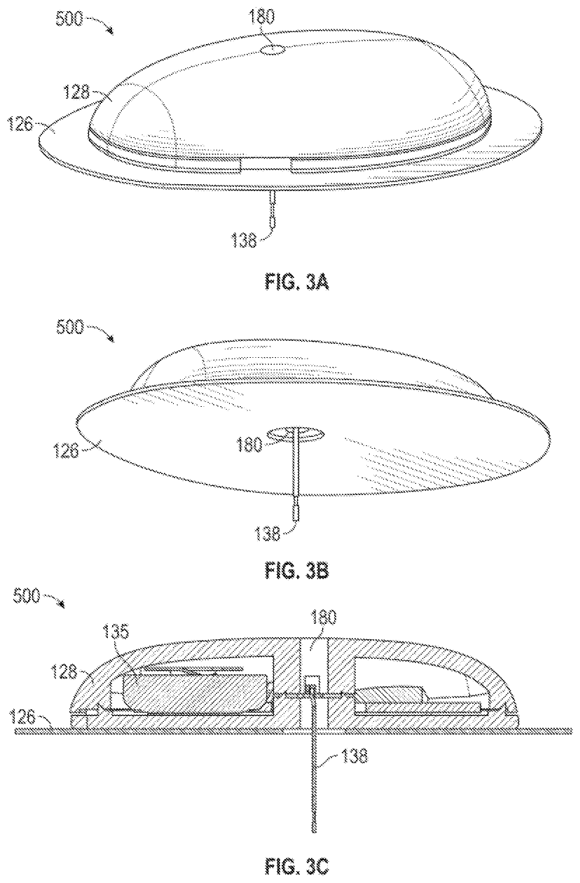

FIGS. 3A, 3B, and 3C illustrate an exemplary implementation of analyte sensor system 101 implemented as a wearable device such as an on-skin wearable medical device or sensor assembly 500. As shown in FIG. 3A, on-skin sensor assembly comprises a body in the form of a housing 128. A patch 126 can couple the housing 128 to the skin of the host. The adhesive of the patch 126 can be a pressure sensitive adhesive (e.g. acrylic, rubber based, or other suitable type) bonded to a carrier substrate (e.g., spun lace polyester, polyurethane film, or other suitable type) for skin attachment. The housing 128 may include a through-hole 180 that cooperates with an applicator such as a sensor inserter device (e.g., a sensor insertion needle, not shown) that is used for implanting sensor 138 under the skin of a host.

The wearable sensor assembly 500 can include electrical components in the form of sensor electronics 112 (e.g., as at least a portion of electronics module 135) operable to measure and/or analyze glucose indicators sensed by glucose sensor 138. Sensor electronics 112 within sensor assembly 500 can transmit information (e.g., measurements, analyte data, and glucose data) to a remotely located device (e.g., 114, 116, 118, 120 shown in FIG. 1). As shown in FIG. 3C, in this implementation sensor 138 extends from its distal end up into through-hole 180 and is routed to electrical components in the form of an electronics module 135 inside the enclosure or housing 128. The working electrode 211 and reference electrode 212 are connected to circuitry in the electronics module 135 which includes the potentiostat.

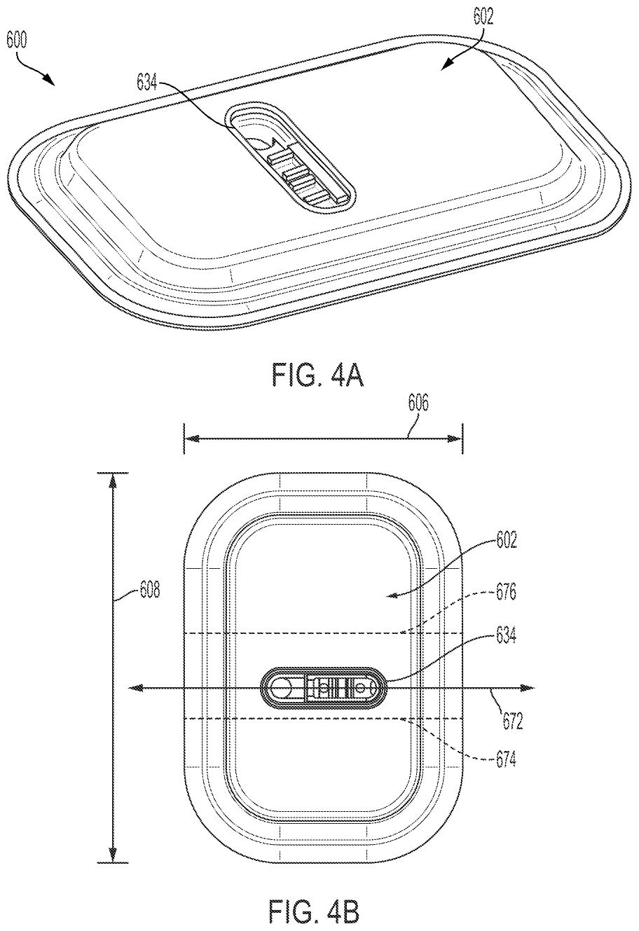

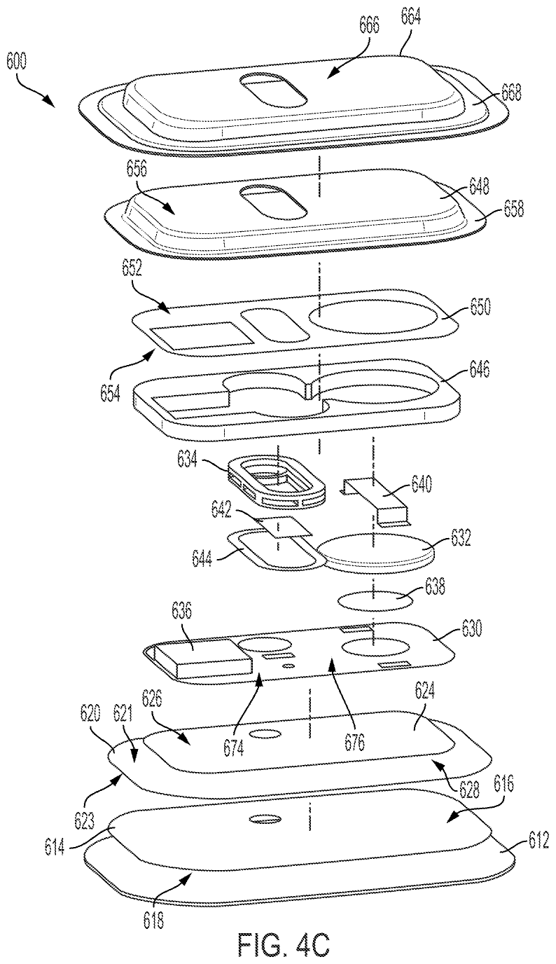

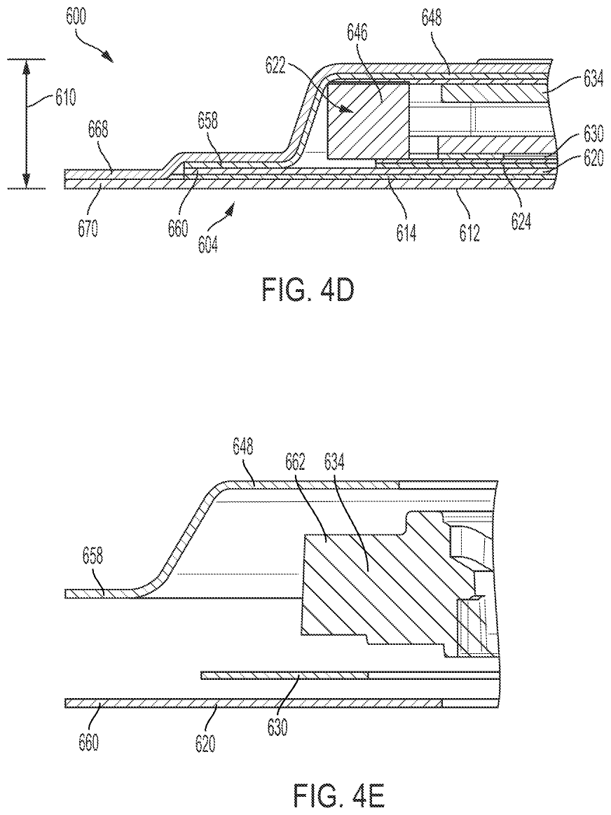

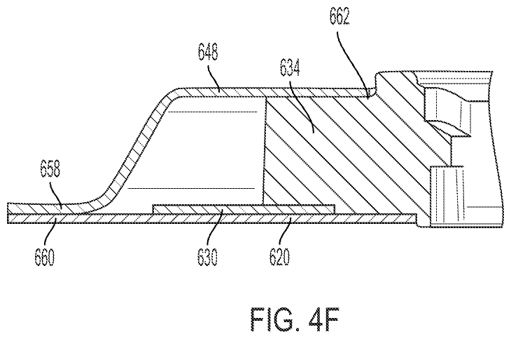

Variations in a configuration of a housing for the on-skin wearable medical device or sensor assembly may be provided. FIGS. 4A-4F, for example, illustrates an implantation including a body in the form of a housing 600 configured to be worn on the skin and configured to couple to an analyte sensor or transcutaneous analyte sensor, the housing 600 including a wall comprising a film layer. The body may be configured to be placed adjacent the skin of a host.

FIG. 4A illustrates a perspective view of the housing 600, showing a top portion 602 raised above a bottom portion 604 (marked in FIG. 4D). The bottom portion 604 may be configured to be proximate the skin of the host and the top portion may be raised from the bottom portion 604 in a direction away from the skin of the host.

The housing 600 may have dimensions including a width 606, and a length 608 (marked in FIG. 4B) and a height 610 (marked in FIG. 4D). The length 608 may be greater than the width 606. The housing 600 may have a rectangular shape as shown in FIG. 4B, or may have another shape (e.g., circular, triangular, hexagonal, or others) as desired. In implementations, the width 606 and the length 608 may each be greater than the height 610. The housing 600 may have a thin plate appearance with the height less than the width 606 and length 608. In implementations, the width 606 or the length 608 may each be less than the height 610.

In implementations, the housing 600 may have a construction including multiple components, or layers of components forming the housing 600 and the on-skin wearable medical device or sensor assembly. The housing 600 or the on-skin wearable medical device or sensor assembly may comprise an assembly of components. FIG. 4C, for example, illustrates an exploded perspective view showing components that may comprise the housing 600 and the on-skin wearable medical device or sensor assembly. In implementations, features disclosed in regard to FIG. 4C may be excluded or substituted as desired.

A bottom portion of the housing 600 may include a patch 612 coupled to the housing 600 and configured to couple the housing 600 to skin of the host. The patch 612, for example, may comprise a flexible material and may be configured to pass moisture therethrough. The patch 612 may allow moisture to pass therethrough to enhance the breathability of the patch 612. Such breathability may reduce adverse effects to the host's skin at the deployment site of the housing 600 (e.g., ability to pass sweat or other moisture therethrough).

In implementations, the patch 612 may have a same outer perimeter size as the housing 600. As such, the patch 612 may not protrude from the outer periphery of the housing 600. FIG. 4D, for example, illustrates the patch 612 extending to the outer periphery of the housing 600. In implementations, the patch 612 may protrude from the outer periphery of the housing 600.

Referring to FIG. 4C, a first adhesive layer 614 may be provided that may couple the patch 612 to the housing 600. The first adhesive layer 614, for example, may comprise dual sided adhesive, with an adhesive on a top surface 616 of the first adhesive layer 614 and on a bottom surface 618 of the first adhesive layer 614. Other configurations of adhesive layers 614 may be utilized as desired.

The first adhesive layer 614 may couple the patch 612 to a bottom film layer 620 of the housing 600. The bottom film layer 620, for example, may comprise a wall of the housing 600 that may seal an interior cavity 622 (marked in FIG. 4D) of the housing 600. The bottom film layer 620 may include a top surface 621 and a bottom surface 623. In implementations, the bottom film layer 620 may be configured to prevent moisture from passing therethrough, to reduce the possibility of moisture entering the interior cavity 622 of the housing. The bottom surface 623 may face towards the skin and at least a portion of the bottom surface 623 may comprise a film layer.

A second adhesive layer 624 may be positioned on the top surface 621 of the bottom film layer 620. The second adhesive layer 624, similar to the first adhesive layer 614, may comprise dual sided adhesive, with adhesive on the top surface 626 of the second adhesive layer 624 and adhesive on the bottom surface 628 of the second adhesive layer 624.

In implementations, the bottom film layer 620 may comprise adhesive surfaces on one or more of the top surface 621 or the bottom surface 623 of the bottom film layer 620 to couple the bottom film layer 620 to the patch 612 or the electrical substrate 630. As such, one or more of the first adhesive layer 614 or the second adhesive layer 624 may be excluded as desired.

The housing 600 may include one or more electrical components positioned therein. The electrical components may be positioned within an interior cavity of the housing. For example, an electrical substrate 630 may be provided that may be configured to electrically couple to one or more other electrical components. In implementations, the electrical substrate 630 may be configured to be flexible to allow for flexibility of the housing 600. In implementations, the electrical substrate 630 may include one or more bending sections that may be configured to allow the housing 600 to bend at the bending sections. The housing 600 may include one or more bending sections as may be disclosed herein. The housing 600 may be flexible.

The electrical substrate 630 may support electrical components thereon, including a power source such as a battery 632, a socket 634 for receiving a plug coupled to an analyte sensor or transcutaneous analyte sensor, and/or sensor electronics 636 for processing a signal received from the analyte sensor or transcutaneous analyte sensor. The sensor electronics 636, for example, may include a processor for processing signals from the sensor and may include a transmitter for transmitting signals to a receiver. The socket 634 may include one or more electrical contacts or terminals for the sensor.

The power source such as the battery 632 may be configured to power the electrical components within the interior cavity 622 (marked in FIG. 4D). In implementations, a conductive tape 638 may be configured to electrically couple the power source to the electrical components. As shown in FIG. 4C, the conductive tape 638 may be positioned to contact a negative terminal of a battery. In implementations, conductive tape 640 may be positioned to contact a positive terminal of a battery. The conductive tape 640 may wrap around at least a portion of the battery 632 to couple to the electrical substrate 630 and provide power to the electrical substrate and the electrical components coupled to the electrical substrate.

The conductive tape 640 may be flexible to enhance the flexibility of the housing 600, and reduce the overall size and stiffness of the housing 600. In implementations, the conductive tape 640 may be omnidirectional and configured to allow current to flow therethrough in a variety of directions. Other forms of conductive tape may be utilized with the housing 600.

Conductive tape 642 may be utilized to electrically couple the socket 634 to the electrical substrate 630. The conductive tape 642 may be uni-directional in embodiments, to allow electrical signals and power from the socket 634 to flow in a desired direction between the electrical substrate 630 and the socket 634. In implementations, the conductive tape 642 may include two sections, with a first section allowing for uni-directional flow to the socket 634 from the electrical substrate 630 and a second section allowing for uni-directional flow from the socket 634 to the electrical substrate 630. The first section and second section may be spaced from each other on a single piece of uni-directional conductive tape to avoid the possibility of electrical interference. In implementations, the conductive tape 642 may include a cut or gap that physically isolates the first section and the second section to avoid electrical interference.

In implementations, other forms of electrical contacts between the socket 634 and the electrical substrate 630 may be utilized. For example, electrical contacts or terminals such as elastomeric pucks or other forms of contacts or terminals may be utilized to connect the socket 634 and the electrical substrate 630. The conductive tape 642 accordingly may be excluded from use in embodiments, or utilized in combination with other forms of electrical contacts. Electrical contacts or terminals as disclosed in implementations herein may be utilized.

Referring to FIG. 4C, a third adhesive layer 644 may be provided that may couple the socket 634 to the electrical substrate 630.

A filler 646 may be positioned within the interior cavity 622 (marked in FIG. 4D) of the housing 600. The filler 646 may comprise a fill layer that is configured to fit between the bottom film layer 620 and the top film layer 648 and may be shaped to fill voids that may surround the electrical components within the housing 600. For example, the filler 646 may be shaped to contour to the power source in the form of the battery 632, and may contour to the socket 634, and may contour to the sensor electronics 636. The filler 646 may include cut-outs contouring to such components, with the voids or rest of the interior cavity 622 filled by the filler 646. The filler 646 may have a height that raises no greater than the height of the electrical components. For example, the height of the filler 646 may be at or less than the height of the socket 634.

In implementations, the filler 646 may comprise a relatively lightweight material and may be configured to not allow for passage of air through the material of the filler 646. For example, the filler 646 may comprise a closed cell foam or other material that does not allow for passage of air. Such a feature may be beneficial if the housing 600 experiences air pressure changes. A material such as closed cell foam may reduce the possibility of expansion of air within the housing 600 according to a reduced air pressure exterior of the housing 600. As such, the possibility of rupture or other damage to the housing 600 may be reduced.

In implementations, the filler 646 may comprise a compliant material that may provide impact protection for the housing 600.

A fourth adhesive layer 650 may be provided that may couple the filler 646 to the top film layer 648. The fourth adhesive layer 650, similar to the first and second adhesive layers, may comprise dual sided adhesive, with adhesive on the top surface 652 of the fourth adhesive layer 650 and adhesive on the bottom surface 654 of the fourth adhesive layer 650. In implementations, the top film layer 648 may comprise a top or bottom adhesive surface, and thus the fourth adhesive layer 650 may be excluded as desired.

The top film layer 648 may comprise a top portion of the housing 600 and may have a similar construction as the bottom film layer 620. The top film layer 648 may have a contoured shape with a raised portion 656 that is raised relative to a flange 658 that extends about the outer periphery of the top film layer 648. The flange 658 may couple to the bottom film layer 620 to seal the interior cavity 622 marked in FIG. 4D.

For example, referring to FIG. 4D, the flange 658 may couple to an outer portion 660 of the bottom film layer 620. The coupling of the top film layer 648 to the bottom film layer 620 may extend around the entirety of the interior cavity 622 and about the entire periphery of the housing 600. The coupling may occur in a variety of manners, including thermal welding, ultrasonic welding, or other forms of welding or coupling. The seal of the interior cavity 622 may be moisture impermeable to reduce the possibility of moisture entering the interior cavity 622.

Referring to FIGS. 4E and 4F, in implementations, the top film layer 648 and the bottom film layer 620 may each couple to the socket 634. For example, the socket 634 may include a peripheral portion 662 that may extend around the central cavity of the socket 634. The peripheral portion 662 may comprise a flattened portion for the top film layer 648 and the bottom film layer 620 to each abut to seal against the socket 634. As such, moisture impermeability may be provided around the socket 634.

Referring back to FIG. 4C, a cover layer 664 may be provided and may comprise an outer top surface of the housing 600. The cover layer 664 may be positioned over the top film layer 648. The outer top surface may be configured to face away from the skin. At least a portion of the outer top surface may comprise a film layer. In implementations, the cover layer 664 may comprise a film layer of the housing 600. In implementations, the cover layer 664 may have other forms. The cover layer 664, similar to the top film layer 648, may include a raised portion 666 that is raised above a flange 668 extending about the periphery of the cover layer 664. The flange 668 may be configured to couple to an outer portion 670 of the patch 612, as shown in FIG. 4D for example.

In implementations, the cover layer 664 may comprise a smooth layer providing a smooth, low friction, outer surface for the housing 600. The cover layer 664 may further provide improved aesthetics for the housing 600.

As discussed, at least a portion of the top portion 602 of the housing 600 may include the film layer, or at least a portion of the bottom portion 604 (marked in FIG. 4D) of the housing 600 may include the film layer. Both the top portion 602 and bottom portion 604 may include the film layer in implementations, and the film layers may be coupled to each other to form a seal of the interior cavity 622.

Each film layer may be flexible in implementations, and as such, the housing 600 may be flexible in implementations. The flexibility of the housing 600 may allow for improved contour to the skin of a host, and for flexibility upon movement of the skin. The housing 600 may have a relatively thin profile, allowing for flexibility in directions both towards and away from the skin.

Referring to FIGS. 4A and 4B, in implementations, the socket 634 may comprise an opening on the top portion 602 of the housing 600. The socket 634 may be exposed and comprise an opening on the top outer surface of the housing 600. The socket 634 may be configured to receive a plug coupled to an analyte sensor or transcutaneous analyte sensor. The plug may be configured to be inserted into the socket 634 in a direction from above the housing 600 downward towards the socket 634. In implementations, the socket 634 may have an oblong shape, with a long dimension 672 of the socket 634 extending along the width 606 of the housing 600. Such an orientation may provide a variety of benefits.

For example, the long dimension 672 of the socket 634 extending along the width 606 of the housing 600 may allow the electronic components (shown in FIG. 4C) to be positioned in areas separate from each other, with the flexible electrical substrate 630 positioned therebetween. The electronic components may be spaced along the length 608 of the housing 600. For example, the sensor electronics 636 may be spaced from the socket 634, which may be spaced from the battery 632. The orientation of the socket 634 along the width 606 of the housing 600 accordingly may allow the housing 600 to bend about the bending sections 674, 676 (marked in FIGS. 4B and 4C) positioned between these respective electronic components. The axes that the housing 600 may bend about may extend parallel with the long dimension 672 of the socket 634 and along the width 606 of the housing 600.

In construction, the use of film layers may allow for high-speed, reel-to-reel manufacturing processes. For example, each of the film layers may comprise a flattened film sheet, which may be thermoformed or otherwise formed into a desired shape. The film layers may roll together and weld together in a manner that forms the housing 600 (e.g., with the top film layer 648 and the bottom film layer 620 being drawn from a reel and being contacted with each other and welded together in a high-speed process). The other layers or components may be die cut and inserted into the layers during assembly. Such a configuration may allow for mass production of the housings 600 and the components contained therein. Other manufacturing processes may be utilized as desired.

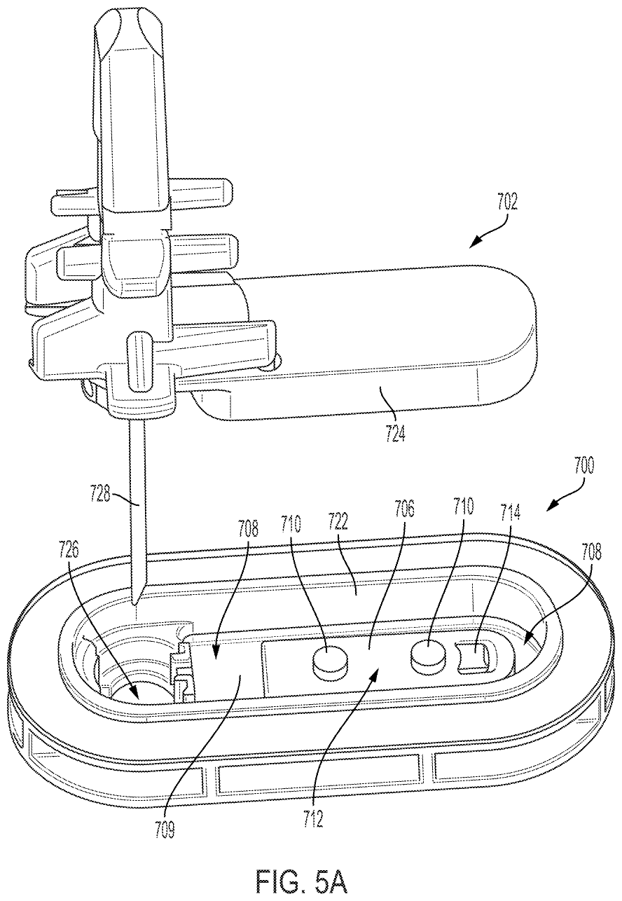

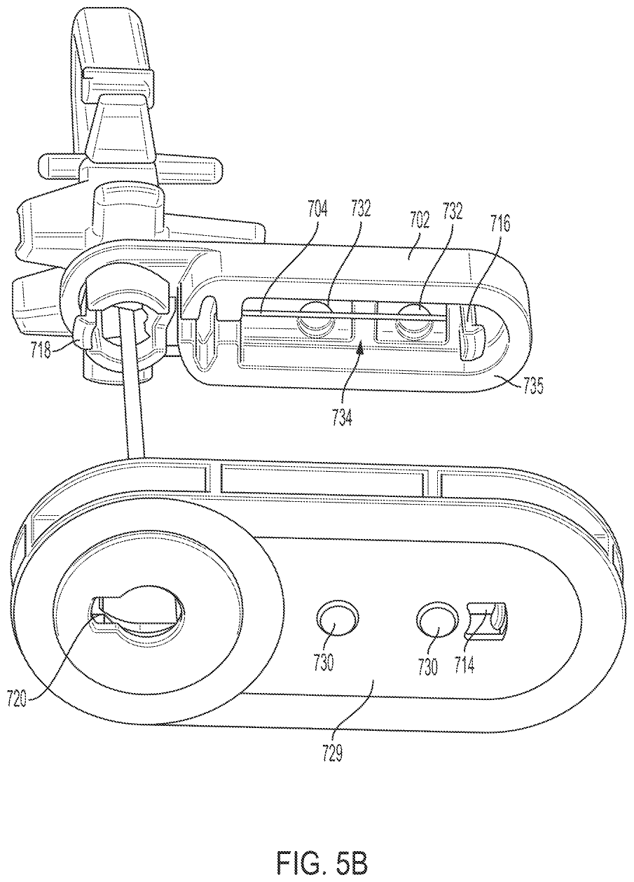

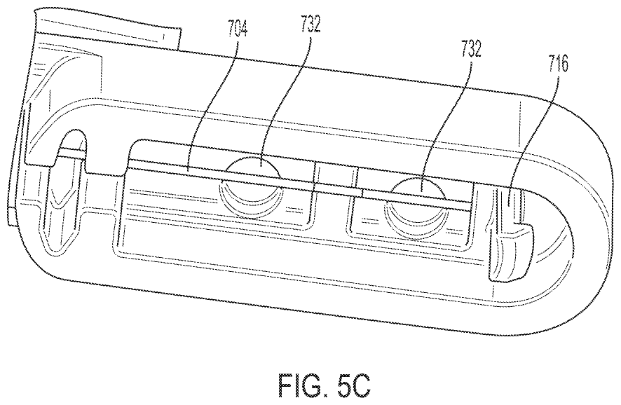

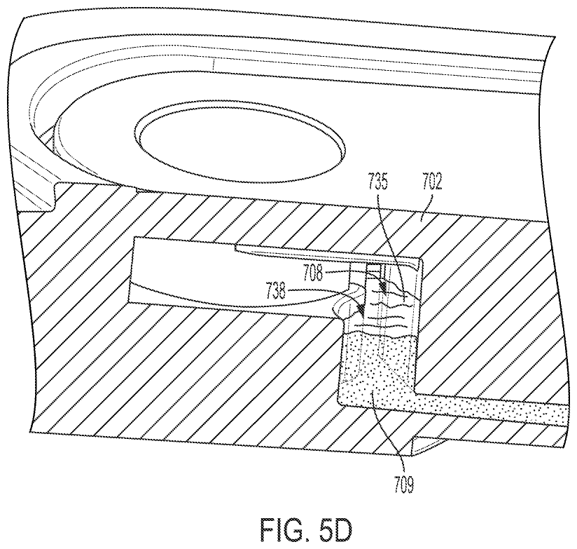

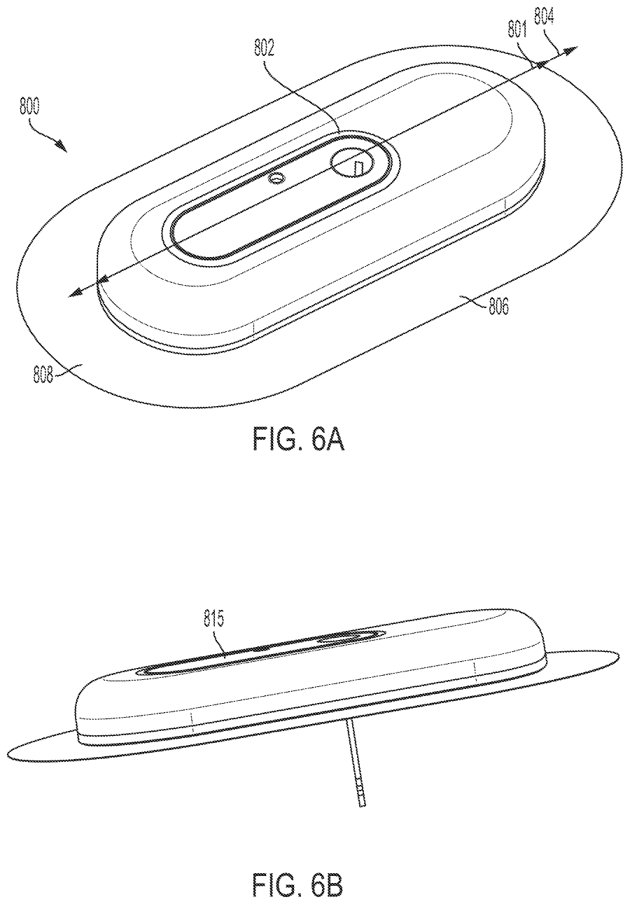

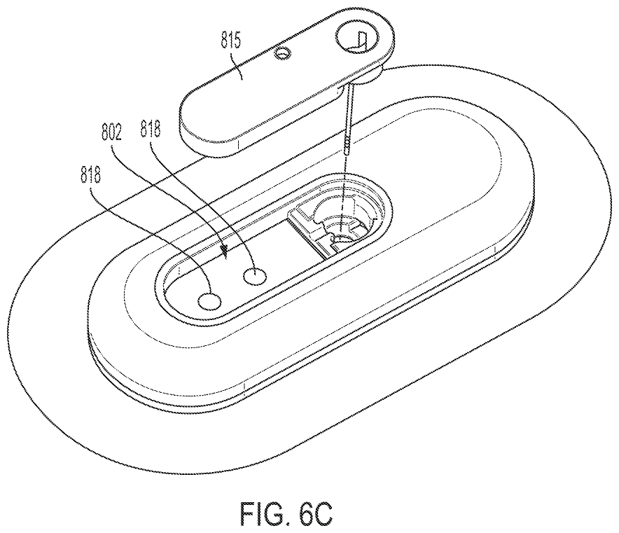

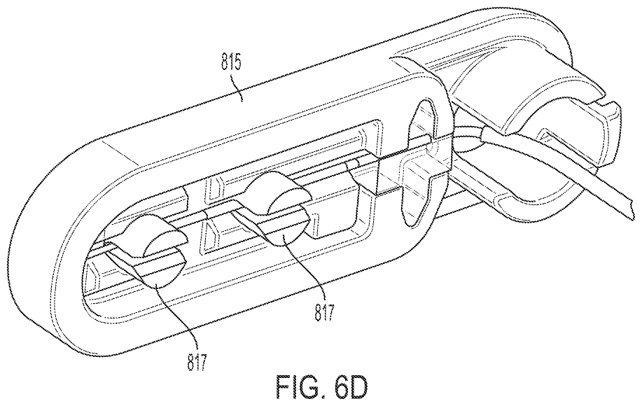

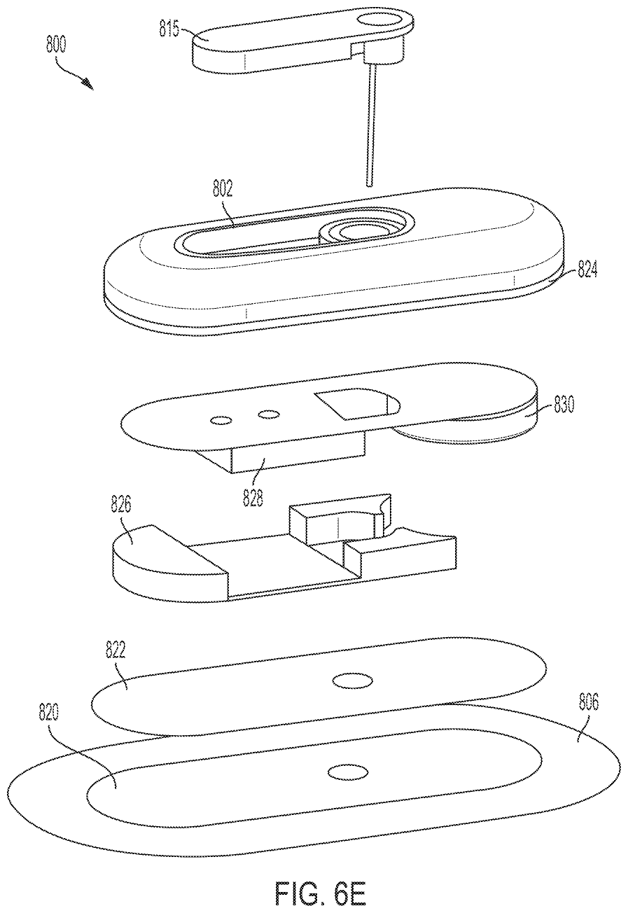

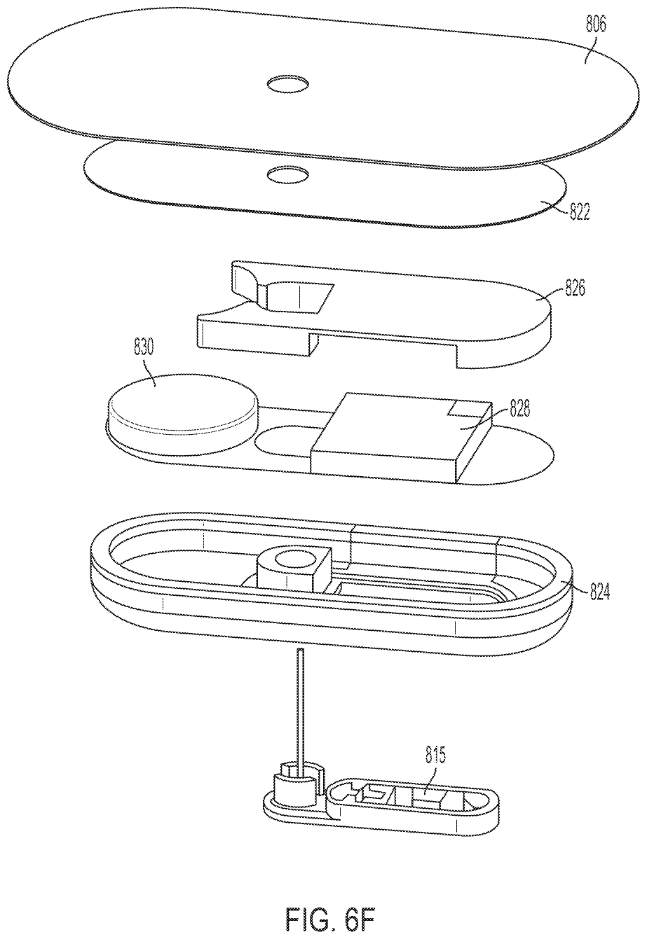

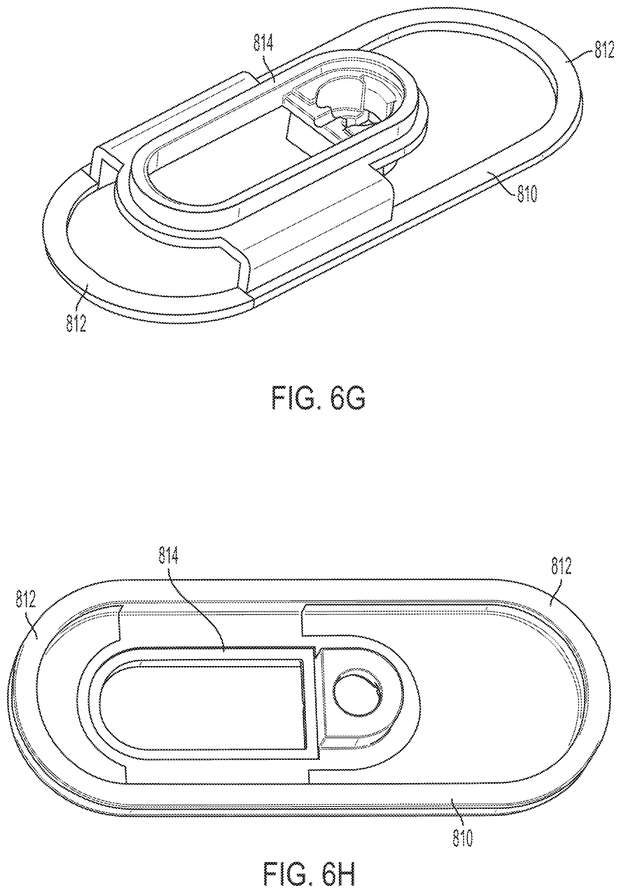

FIGS. 5A-5D illustrate an embodiment of a socket 700 and a plug 702 that may be utilized solely or in combination with implementations herein. The socket 700, for example, may be utilized in implementations having a socket, such as the implementations of FIGS. 4A-4F, or the implementations of FIGS. 6A-8F.

Referring to FIG. 5A, the socket 700 may be configured to be coupled to a body that may be worn on the skin, such as implementations of housings disclosed herein or another form of body. The socket 700 may be configured to couple to the plug 702 that may be coupled to an analyte sensor 704 or transcutaneous analyte sensor as marked in FIG. 5B for example.

The socket 700 may include a raised portion 706 and a channel 708 surrounding the raised portion 706. The raised portion 706 may include one or more electrical contacts 710 for electrical connection with the analyte sensor 704 and the channel 708 may include a fluid 709 disposed therein for forming a seal with at least a portion of the plug 702.

The raised portion 706 may be raised above the channel 708 and may include a top surface 712 that may have a flattened shape. The flattened shape of the top surface 712, for example, may allow the electrical contacts 710 to protrude from the top surface 712 for connection with the analyte sensor 704. The electrical contacts 710 may be positioned on the top surface 712 that is configured to face the plug.

The raised portion 706 may include a first coupler 714 in the form of an aperture for receiving a corresponding second coupler 716 of the plug 702 (marked in FIG. 5B). The first coupler 714 may receive the corresponding second coupler 716 to mechanically couple the plug 702 to the socket 700. The plug 702 may include an additional third coupler 718 (marked in FIG. 5B) that may couple to a fourth coupler 720 in the form of an aperture for receiving the third coupler 718. The second coupler 716 and third coupler 718 may comprise protrusions for engaging the apertures of the first and fourth couplers 714, 720, although other configurations may be utilized in embodiments. For example, one or more of the second coupler 716 and third coupler 718 may comprise apertures and one or more of the first and fourth couplers 714, 720 may comprise protrusions, although other configurations of couplers may be utilized as desired.

Referring back to FIG. 5A, the channel 708 may comprise a lowered portion of the socket 700 and may be bound by a raised outer wall 722 surrounding the channel 708. The raised outer wall 722 may be configured to retain the fluid 709 within the channel 708. The raised outer wall 722, in implementations, may have a greater height than the raised portion 706, which may allow for alignment between the raised outer wall 722 and the plug 702. For example, the raised outer wall 722 may contour to the shape of the peripheral surface 724 of the plug 702 to allow for alignment and sealing between the plug 702 and the socket 700.

The socket 700 may further include an aperture 726 for the analyte sensor 704 to pass through, and for the insertion needle 728 to pass through.

The fluid 709, in implementations, may comprise a fluid for sealing the connection between the socket 700 and the plug 702 to reduce the possibility of moisture interfering with the electrical connection between the electrical contacts 710 and the analyte sensor 704. The fluid may be configured to be moisture impermeable, and as such may comprise a gel. The gel may comprise petroleum jelly or other forms of gel or fluids as desired. The fluid may be disposed within the channel 708 and configured to be displaced upon the plug 702 entering the socket 700.

The electrical contacts 710 may protrude from the top surface 712 and in implementations may extend through the socket 700 to pass to a bottom surface 729 (marked in FIG. 5B) facing opposite the top surface 712. The electrical contacts 710 may include portions 730 that protrude from the bottom surface 729 and may be configured to electrically couple to electrical components, such as an electrical substrate or other electrical components. The electrical components, for example, may comprise a sensor electronics for receiving a signal from the analyte sensor 704 or a power source. Other forms of electrical components may be utilized.

In implementations, the electrical contacts 710 may comprise an electrically conductive elastomeric material. Such material may allow for compression of the electrical contacts 710 upon contact and compression by the analyte sensor 704. Other forms of electrical contacts may be utilized in implementations.

Referring to FIG. 5C, the plug 702 may include recesses 732 for electrically conductive portions of the analyte sensor 704 to be positioned within and for the electrical contacts 710 to enter. The recesses 732, for example, may be shaped to receive the electrical contacts 710 and allow for electrical connection between the contacts 710 and the analyte sensor 704. Portions of the analyte sensor 704 that may be contacted may include a respective working electrode and reference electrode, although other portions may be contacted as desired.

The plug 702 may include a cavity 734 for the raised portion 706 of the socket 700 to enter into. The plug 702 may include an outer wall 735 for entering into the channel 708 and surrounding the raised portion 706.

In operation, the plug 702 may be inserted into the socket 700 to electrically connect the electrical contacts 710 with the analyte sensor 704. Upon insertion, the fluid 709 may be displaced due to the presence of the outer wall 735. FIG. 5D, for example, illustrates the outer wall 735 positioned within the channel 708.

In implementations, the socket 700 may include a reservoir 738 for receiving and storing any excess amount of the fluid 709 following the coupling of the plug 702 to the socket 700. The reservoir 738 may reduce the possibility of the fluid 709 leaking upon insertion of the plug 702 into the socket 700.

The configuration of plug 702 and/or socket 700 may be utilized with any implementation disclosed herein.

FIGS. 6A-6I illustrate an implantation of an on-skin wearable medical device or on-skin sensor assembly including an elongate housing 800 having a long dimension 801, and an elongate socket 802 having a long dimension 804 extending along the long dimension 801 of the elongate housing 800.

Referring to FIG. 6A, the elongate housing 800 may be configured similarly as the housing 600 shown in FIG. 4A unless stated otherwise. For example, the electrical components within the elongate housing 800 may be comprise similar electrical components as the electrical components within the housing 600.

The elongate housing 800 may have an oblong shape and may be configured to be worn on the skin. For example, a patch 806 may be utilized to couple the elongate housing 800 to the skin. The patch 806 may be coupled to a bottom portion of the elongate housing 800 and may be configured to couple to elongate housing 800 to skin. The patch 806 may protrude outward from the outer periphery of the elongate housing 800 to form a skirt portion 808 extending radially outward from the elongate housing 800.

The elongate housing 800 may be constructed of one or more materials which may be rigid or may be flexible. In implementations, the elongate housing 800 may be constructed of co-molded materials comprising a first material having a greater stiffness than a second material. For example, referring to FIG. 6G, a first portion of the elongate housing 800 may comprise a frame 810 constructed of the first material. The frame 810 accordingly may comprise a relatively rigid body, including an outer peripheral loop 812 and a central portion 814 for supporting the socket 802. FIG. 6H illustrates a bottom perspective view of the frame 810.

The second material 816 (marked in FIG. 6I) may be co-molded upon the frame 810 and may comprise a flexible material. The second material 816 may be coupled to the frame 810. The second material 816 may form at least a portion of an outer surface of the elongate housing 800. The second material 816 may improve the compliance of the elongate housing 800 and may allow the elongate housing 800 to be flexible, to contour to the shape of the host's skin and/or movement of the host's skin. Proportions of the elongate housing 800 comprising the rigid first material and the compliant second material may be varied as desired to produce a desired flexibility for the elongate housing 800.

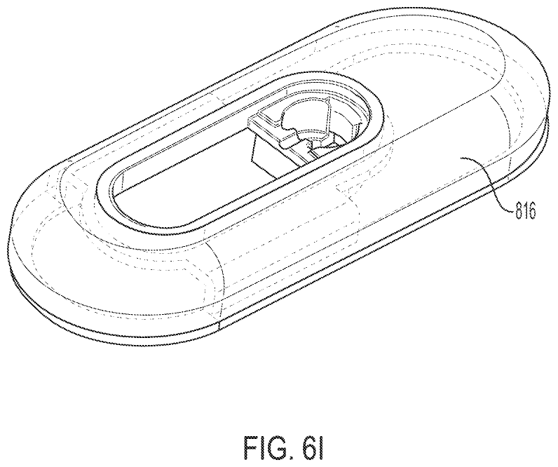

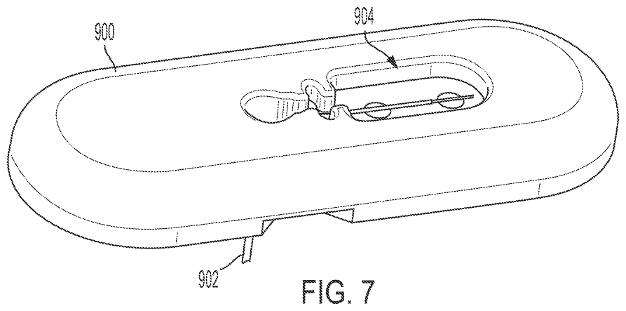

FIG. 7, for example, illustrates an embodiment in which an outer housing 900 is entirely constructed of a rigid material to form a rigid outer housing 900.

Referring to FIGS. 6B and 6C, the socket 802 may be configured to receive a plug 815 in a similar manner as other couplings of sockets and plugs disclosed herein. Referring to FIG. 6D, the plug 815 may be configured similarly as the plug 702, yet may include protruding electrical contacts 817 for coupling with the flattened electrical contacts 818 of the socket 802 shown in FIG. 6C.

The elongate housing 800 may include a multi-level construction that may reduce the overall width and length of the elongate housing 800 from a configuration as shown in FIGS. 4A-4F. However, the multi-level construction may increase the height of the elongate housing 800 from a configuration as shown in FIGS. 4A-4F.

FIG. 6E, for example, illustrates a top perspective exploded view of the elongate housing 800. The elongate housing 800 may include the patch 806, and an adhesive layer 820 for coupling the patch 806 to a bottom film layer 822. The bottom portion of the elongate housing may include a film layer. The bottom film layer 822 may be constructed similarly as the bottom film layer 620 of FIGS. 4A-4F. The bottom film layer 822 may couple to an outer periphery 824 (marked in FIG. 6F) of the elongate housing 800 to seal the interior cavity of the elongate housing 800.

The elongate housing 800 may include a filler 826, which may be configured similarly as the filler 646 of FIGS. 4A-4F. The multi-level construction of the elongate housing 800 is shown with regard to the electrical components. The sensor electronics 828 including the transmitter, for example, may be positioned directly below the socket 802 and the plug 815. The socket 802 may be positioned above the transmitter. The sensor electronics 828 may comprise a lower level of the elongate housing 800 with the elongate socket 802 comprising an upper level of the elongate housing 800 positioned above the lower level. The elongate socket 802 is accordingly positioned above the sensor electronics 828 and transmitter in a multi-level configuration. In addition, the power source such as a battery 830 may be positioned in the lower level with the sensor electronics 828.

FIG. 6F illustrates an inverted view of the elongate housing 800 from the configuration shown in FIG. 6E.

The multi-level construction may reduce the overall footprint, or length and width of the elongate housing relative to a configuration as shown in FIGS. 4A-4F for example. An increase in height, however, may result.

Referring to FIG. 6A, the extension of the long dimension 804 of the elongate socket 802 along the long dimension 801 of the elongate housing 800 may further reduce the overall footprint, or length and width of the elongate housing. This is in contrast to an implementation as shown in FIGS. 4A-4F, in which the long dimension 672 of the socket 634 extends perpendicular to the long dimension of the housing 600. As such, reduced footprint of the elongate housing 800 may result.

The configuration of the elongate housing 800 or elongate socket 802 may be utilized with any implementation disclosed herein.

In implementations, the transcutaneous analyte sensor may be in a fixed configuration relative to the housing. FIG. 7, for example, illustrates the analyte sensor 902 fixed relative to the housing 900. The housing 900 yet may retain a sensor connection region 904 on a top portion or top outer surface of the housing 900. Various modifications of implementations may be provided.

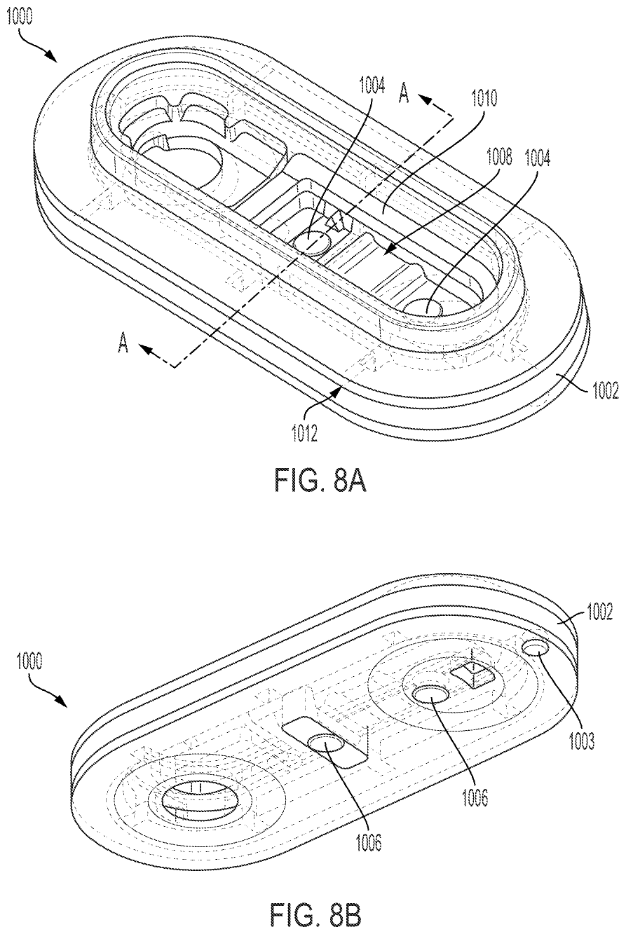

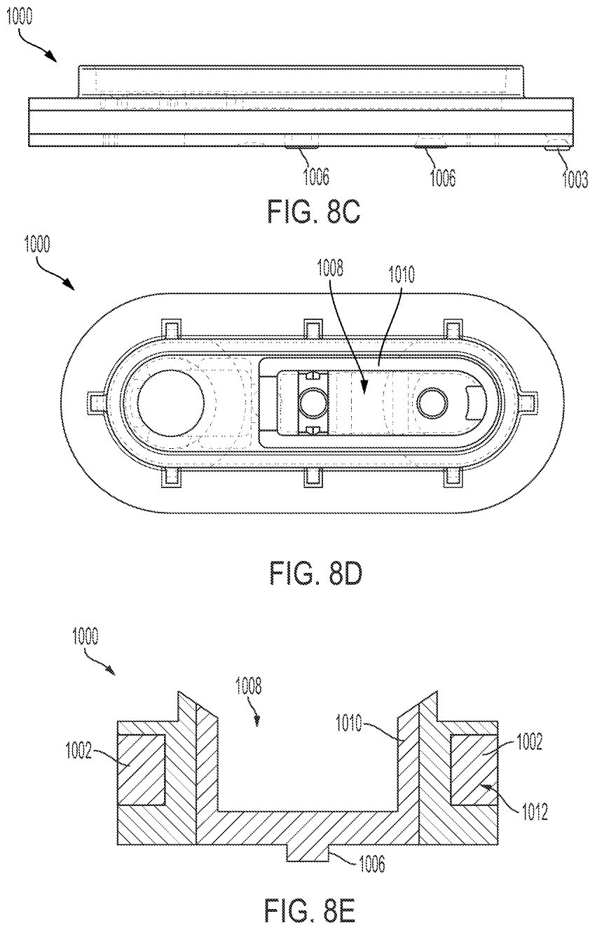

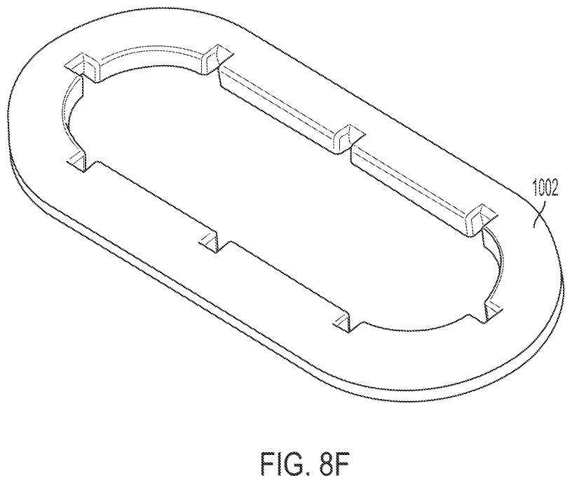

FIGS. 8A-8F illustrate an implementation of a socket 1000 including a loop antenna 1002 surrounding the socket 1000. The socket 1000 may be configured similarly as other implementations of sockets disclosed herein, unless stated otherwise.

The socket 1000 may include electrical contacts 1004 that may be configured similarly as other embodiments of electrical contacts disclosed herein. For example, the electrical contacts 1004 may be configured for electrical connection with an analyte sensor or otherwise for connection with one or more electrical contacts coupled to a plug. The electrical contacts 1004 may comprise an electrically conductive elastomeric material such as elastomeric pucks or another form of electrical contact. The electrical contacts 1004 may pass through the body of the socket 1000 to form portions 1006 on a bottom surface of the socket 1000 as shown in FIG. 8B for example.

The electrical contacts 1004 may be positioned within a cavity 1008 of the socket 1000.

A gasket 1010 may be positioned within the cavity 1008 of the socket 1000 and may be configured to form a seal with a plug that may be inserted into the socket 1000. The plug, for example, may be configured similarly as implementations of plugs disclosed herein. The gasket 1010, for example, may surround an outer surface of the plug to form the seal. The gasket 1010 may comprise an elastomeric material, and in implementations, may comprise a continuous body with at least one of the electrical contacts 1004 (as shown in the cross-sectional view of FIG. 8E).

The socket 1000 may include a channel 1012 that surrounds the socket 1000. The channel 1012 may surround the cavity 1008 of the socket 1000 and the electrical contacts 1004. FIG. 8C illustrates a side view of the socket 1000 and FIG. 8D illustrates a top view of the socket 1000.

The loop antenna 1002 may surround the socket 1000 and may be positioned within the channel 1012. An isolated view of the loop antenna 1002 is shown in FIG. 8F. The loop antenna 1002 may form an entire loop around the socket 1000 or in implementations may form only a partial loop around the socket 1000 (or at least a partial loop around the socket 1000). Referring to FIG. 8A, the loop antenna 1002 may surround the electrical contacts 1004 within the socket 1000. The loop antenna 1002 may be coupled to the body and configured to receive or transmit signals from the body.

Referring to FIG. 8B, the loop antenna 1002 may include an electrical contact 1003 that may be positioned on a bottom surface of the socket 1000. The electrical contact 1003 may contact electrical components such as an electrical substrate or a power source or may be in electrical connection with sensor electronics. The electrical contact 1003 may be configured to receive electrical signals from the loop antenna 1002 or transmit electrical signals from the loop antenna 1002.

The loop antenna 1002 may have an oblong or elongated shape, which may match a shape of the socket 1000 and the channel 1012. Other shapes of loop antennas may be utilized as desired. FIG. 8E illustrates a cross-sectional view of the loop antenna 1002 positioned within the channel 1012. The loop antenna 1002 may have a rectangular cross section, or may have another cross-sectional shape as desired.

The use of the loop antenna 1002 may beneficially provide a larger overall length of material to be utilized as an antenna. A position extending around the socket 1000 may make use of the oblong shape of the socket 1000 and the space around the cavity 1008 for receiving the plug. Further, in an implementation in which the socket 1000 is positioned at a top portion of a housing, or comprises a top opening in a top outer surface of a housing, the antenna may beneficially be positioned away from the skin of the host, which may improve operation of the antenna. For example, in an implementation as shown in FIG. 6E, a socket may be positioned at an upper level, which may increase the distance from the host's skin.

In implementations, a loop antenna may be utilized elsewhere within a body such as a housing configured to be worn on the skin and configured to couple to a transcutaneous analyte sensor. The loop antenna may be coupled to the body and configured to receive signals or transmit signals from the body. The loop antenna may be provided in a variety of positions within a body or housing. In implementations the loop antenna may be positioned at a top portion or upper level of a housing. Other positions may be utilized.

Any implementation disclosed herein may utilize a loop antenna.

Implementations as disclosed herein may include electrical and mechanical connections between an analyte sensor or transcutaneous analyte sensor and a body. The electrical and mechanical connections may be utilized to transmit power and/or sensor signals to or from the analyte sensor. The electrical and/or mechanical connections may comprise sensor connections. The electrical and/or mechanical connections may comprise a sensor carrier or sensor interposer.

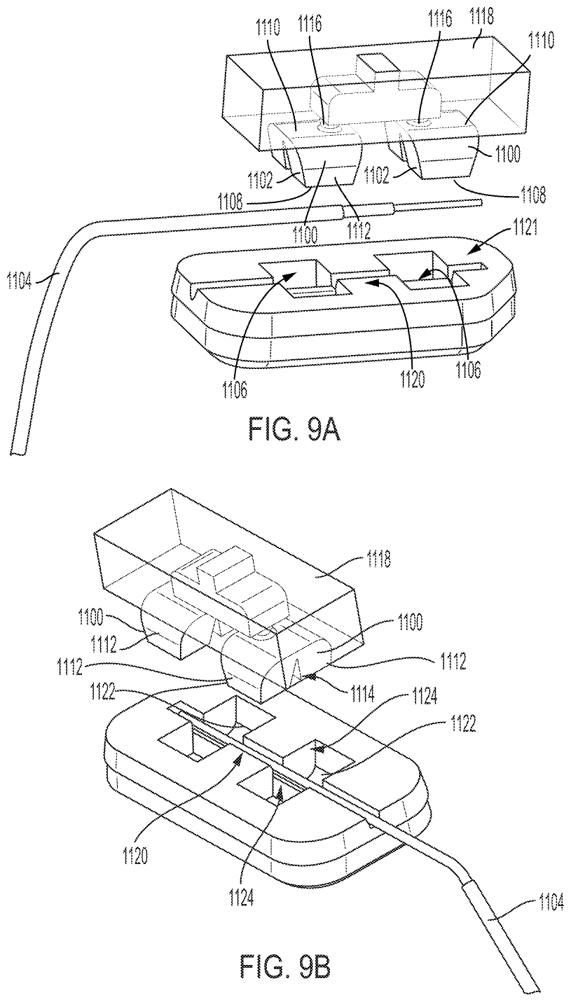

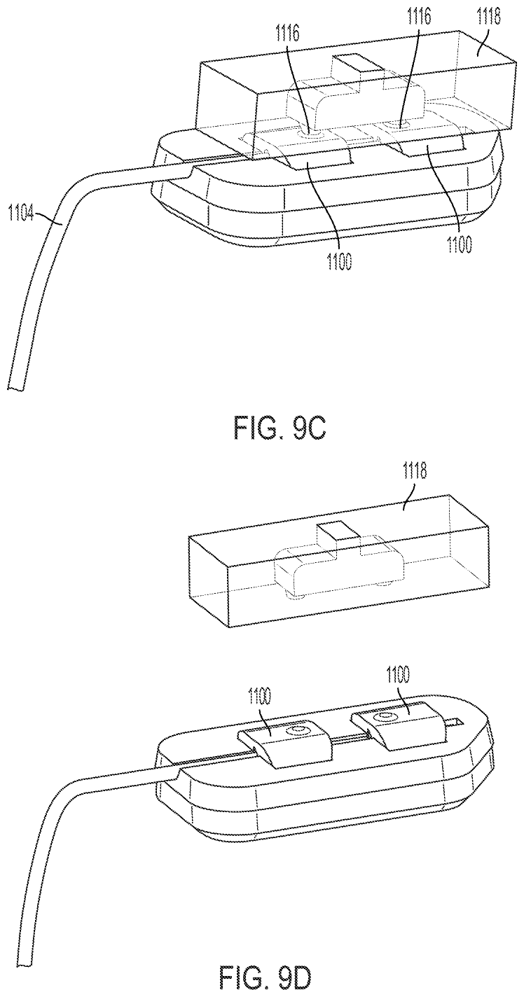

FIGS. 9A-9D, for example, illustrate an implementation including one or more conductor bodies 1100, each having a forked portion 1102 configured to couple to a portion of the analyte sensor 1104, and each configured to be pressed into a respective slot 1106 to couple to the respective portion of the analyte sensor 1104.

The conductor bodies 1100 may include a lower surface 1108 and an upper surface 1110. The lower surface 1108 may include the forked portion 1102. The forked portion 1102 may be formed by arms 1112 of the conductor bodies 1100 being separated from each other by a gap 1114 (marked in FIG. 9B). The arms 1112 may be at an angle relative to each other to form a wedge shape that receives the analyte sensor 1104.

The upper surface 1110 may include a releasable portion 1116 configured to release from an applicator 1118 for applying the one or more conductor bodies 1100 to a sensor receiving portion 1121 of a body. The releasable portion 1116 may comprise a breakable portion in implementations. For example, the applicator 1118 may be configured to twist relative to the releasable portion 1116 such that a shearing force breaks the portion 1116 and releases the conductor bodies 1100 from the applicator 1118 (as represented in FIG. 9D for example).

The one or more conductor bodies 1100 may be configured to be inserted into a sensor receiving portion 1121 of a body. The sensor receiving portion 1121 may comprise a portion of a housing or plug, or other form of body for receiving the analyte sensor 1104. For example, implementations of plugs or housings may utilize a sensor receiving portion 1121 as disclosed herein. The sensor receiving portion 1121 may be positioned on a top portion, or top surface of a housing, or on a bottom portion, or bottom surface of a housing as desired.

The sensor receiving portion 1121 may include the one or more slots 1106 and may include a channel 1120 extending transverse to the one or more slots 1106. The channel 1120 may be configured to receive the analyte sensor 1104 with the analyte sensor 1104 extending along the length of the channel 1120.

The one or more slots 1106 may each include a lower surface 1122 (marked in FIG. 9B) and the channel 1120 may be raised above the lower surface 1122. The one or more slots 1106 may each include a cavity 1124 extending downward from the channel 1120 to a respective lower surface 1122.

Referring to FIG. 9B, in assembly, the analyte sensor 1104 may be inserted into the channel 1120. The applicator 1118 may be coupled to the conductor bodies 1100 and may move the conductor bodies 1100 towards the analyte sensor 1104.

The applicator 1118 may press the arms 1112 of the conductor bodies 1100 into the cavities 1124 and may cause the conductor bodies 1100 to apply a pressure to the analyte sensor 1104 to retain the analyte sensor 1104 in place. FIG. 9C, for example, illustrates the applicator 1118 having pressed the conductor bodies 1100 into place. The conductor bodies 1100 may apply pressure to the analyte sensor 1104 from a vertical direction and from a lateral direction relative to the analyte sensor 1104.

The releasable portions 1116 may then be released via a shearing force or the like provided by the applicator 1118. FIG. 9D illustrates the conductor bodies 1100 in position.

The conductor bodies 1100 may be utilized to electrically couple the analyte sensor 1104 to one or more electrical components. The electrical components may comprise any of the components disclosed herein, and may be positioned within a body of an on-skin wearable medical device or on-skin sensor assembly. The electrical components may comprise an electrical substrate, or sensor electronics, or a power source (e.g., a battery), among other forms of electrical components. The electrical components may be positioned within a body.

The conductor bodies 1100 and configuration of the sensor receiving portion 1121 may be utilized with any implementation disclosed herein.

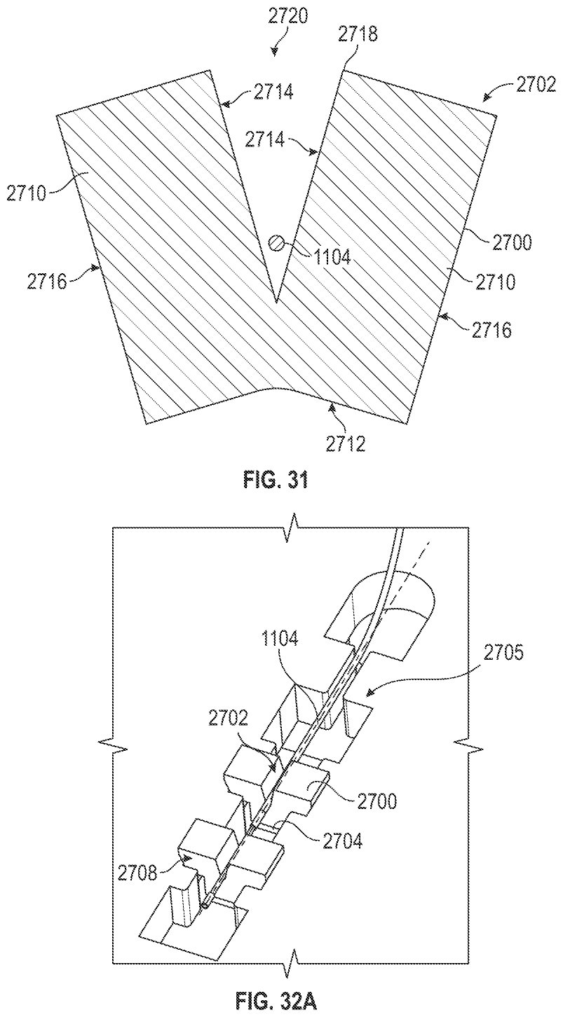

The configuration of the conductor bodies may be varied in implementations. For example, referring to FIGS. 31-33, one or more conductor bodies 2700 may include forked portions 2702 configured to face opposite the lower surface 2704 of a sensor receiving portion 2705 when the conductor bodies 2700 are pressed into a respective slot 2708.

Referring to FIG. 31, a cross-sectional view of the conductor body 2700 is shown relative to the analyte sensor 1104. The conductor body 2700 is shown in a configuration prior to insertion into the respective slot 2708 of the sensor receiving portion 2705. The forked portion 2702 is formed by a plurality or pair of arms 2710 extending from a vertex portion 2712 of the conductor body 2700. The vertex portion 2712 may be coupled to the forked portion 2702.

The arms 2710 may each include an interior surface 2714. The interior surfaces 2714 may be configured to face towards each other and may receive the analyte sensor 1104 between the interior surfaces 2714. The arms 2710 may each include an exterior surface 2716 facing opposite the respective interior surface 2714. The interior surfaces 2714 may extend away from the vertex portion 2712 to an opening 2718 of a channel 2720 for receiving the analyte sensor 1104.

In the configuration shown in FIG. 31, the arms 2710 are flared outward from each other, to form a wedge shape of the forked portion 2702. The conductor body 2700 may be configured to receive the analyte sensor 1104 in such a configuration.

The conductor body 2700 may be extruded or otherwise formed in the configuration shown in FIG. 31.

In implementations, the vertex portion 2712 may comprise a living hinge. The vertex portion 2712, for example, may be configured to deflect such that the arms 2710 may be moved towards each other to close or reduce the size of the channel 2720. The analyte sensor 1104 may be compressed by the interior surfaces 2714 and retained in position by the compression against the analyte sensor 1104. Mechanical and electrical contact with the analyte sensor 1104 may result.

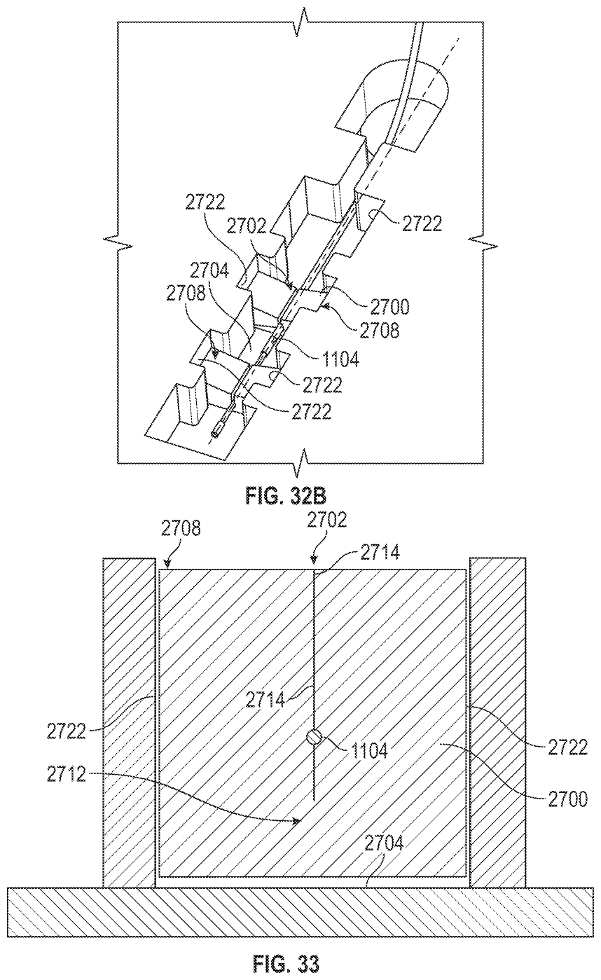

Referring to FIG. 32A, for example, the analyte sensor 1104 may be positioned within one or more conductor bodies 2700 and pressed towards the lower surface 2704. The forked portions 2702 as shown in FIG. 32A may remain flared outward due to the diameter of the analyte sensor 1104. The conductor bodies 2700 and the analyte sensor 1104 may be pressed downward.

FIG. 32B illustrates the conductor bodies 2700 pressed into the slots 2708. An outer wall 2722 of the respective slot 2708 bounding the slot 2708 (and extending upward from the lower surface 2704) may compress the exterior surfaces 2716 of the arms 2710 inward. The analyte sensor 1104 may be compressed laterally within the channel 2720. The vertex portion 2712 of the respective conductor body 2700 faces towards the lower surface 2704 when the conductor bodies 2700 are pressed into the respective slots 2708.

FIG. 33 illustrates a cross-sectional view of a resulting configuration of a conductor body 2700. The interior surfaces 2714 may face towards each other and extend parallel with each other when the conductor body 2700 is pressed into the slot 2708. The interior surfaces 2714 move towards each other upon the conductor body 2700 being pressed into the slot 2708. The vertex portion 2712 may form a bottom or lower portion of the conductor body 2700 that may support the analyte sensor 1104. The conductor bodies 2700 may otherwise have a similar construction as the conductor bodies 1100 discussed in regard to FIGS. 9A and 9B, and may comprise elastomeric bodies or other forms of bodies. The features of FIGS. 31-33 may be utilized with any implementation disclosed herein.

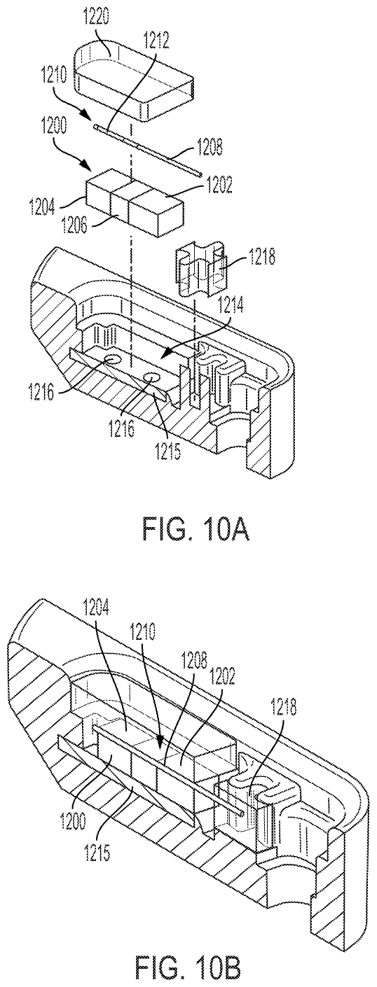

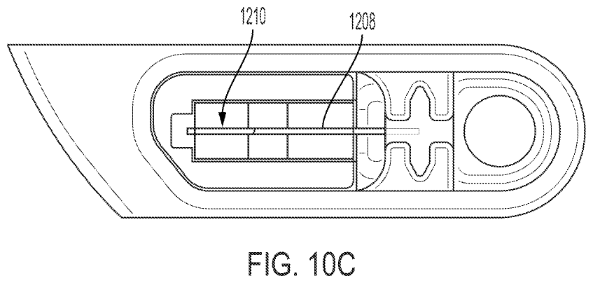

FIGS. 10A-10C illustrate an implementation of a sensor connection or sensor carrier or interposer including an electrical connector body 1200 including a first end portion 1202, a second end portion 1204, and a central portion 1206 positioned between the first end portion 1202 and the second end portion 1204.

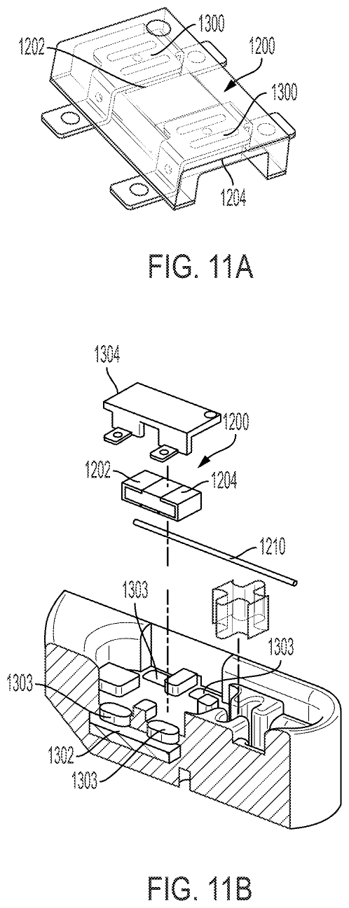

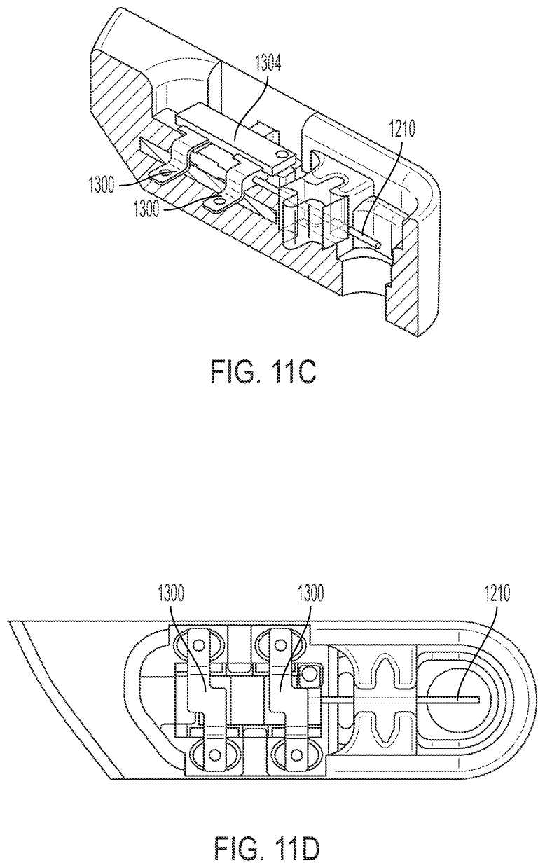

The first end portion 1202 may be conductive and configured to form an electrical connection with a first portion 1208 of an analyte sensor 1210 or transcutaneous analyte sensor as disclosed herein. Only a portion of the analyte sensor 1210 is shown in FIGS. 10A-11D, yet the full analyte sensor 1210 may be configured similarly as other embodiments disclosed herein.

The second end portion 1204 may be conductive and configured to form an electrical connection with a second portion 1212 of the analyte sensor 1210. The first portion 1208 or second portion 1212 of the analyte sensor 1210 may comprise a working electrode or reference electrode as desired, among other portions of the analyte sensor 1210. The first end portion 1208 and the second end portion 1204 may each be configured to electrically connect the sensor 1210 with one or more electrical components of the body.

The central portion 1206 may comprise an insulator electrically insulating the first end portion 1202 from the second end portion 1204.

The electrical connector body 1200 may comprise an elastomeric material, and the first end portion 1202 and the second end portion 1204 each may comprise an electrically conductive elastomeric material. The central portion 1206 may comprise an elastomeric material that comprises an insulator, and the conductive materials of the first end portion 1202 and the second end portion 1204 may be disposed upon the insulator material. The electrical connector body 1200 may comprise a continuous body or continuous elastomeric body extending from the first end portion 1202 to the second end portion 1204.

The electrical connector body 1200 may be positioned within a cavity 1214 comprising a sensor receiving portion. The cavity 1214 may include an electrical substrate 1215 and electrical contacts 1216 for contact with the respective first end portion 1202 and second end portion 1204.

A retainer 1218 in the form of a curable material may be positioned adjacent to the cavity 1214 and configured to retain a portion of the analyte sensor 1210.

A top member 1220 may be provided that may be disposed on the analyte sensor 1210 and the electrical connector body 1200. The top member 1220 may comprise a curable material that may be dispensed upon the electrical connector body 1200 and cured in position to seal the cavity 1214 and the analyte sensor 1210 and the electrical connector body 1200. In implementations, an additional adhesive material may create a seal between the top member 1220 and the cavity 1214 or other body.

In assembly, the electrical connector body 1200 may be positioned upon the electrical contacts 1216 and electrically connected with the electrical substrate 1215. The analyte sensor 1210 may then be positioned upon the electrical connector body 1200, with the portions 1208, 1212 of the analyte sensor 1210 aligned with the respective end portions 1202, 1204 of the electrical connector body 1200. The top member 1220 may then be positioned upon the analyte sensor 1210 and electrical connector body 1200 to press the analyte sensor 1210 against the electrical connector body 1200.

FIG. 10B illustrates a perspective cross-sectional view of a resulting configuration. FIG. 10C illustrates a top view of a resulting configuration.

In implementations, the analyte sensor 1210 may be positioned between the electrical connector body 1200 and the electrical substrate 1215.

Any implementation disclosed herein may utilize such a sensor connection for an analyte sensor.

FIGS. 11A-11D illustrate a variation in the implementations of FIGS. 10A-10C in which conductive tabs 1300 and conductive pads 1303 are utilized. Referring to FIG. 11A, the electrical conductor body 1200 of FIGS. 10A-C may be provided, and conductive tabs 1300 may each electrically connect a respective end portion 1202, 1204 to an electrical substrate 1302 (marked in FIG. 11B). The conductive tabs 1300 may extend over the end portions 1202, 1204 and may secure the end portions in position. The analyte sensor 1210 (shown in FIG. 11B) may be positioned between the electrical conductor body 1200 and the electrical substrate 1302. In implementations, the analyte sensor 1210 may be positioned between the electrical conductor body 1200 and the top member 1304.

The conductive pads 1303 may be positioned to the sides of the electrical conductor body 1200 and may be configured to electrically couple to a respective one of the conductive tabs 1300.

FIG. 11B illustrates an assembly view of the connection. The top member 1304 may be positioned upon the electrical conductor body 1200 in a similar manner as the top member 1220 shown in FIGS. 10A-10C.

FIG. 11C illustrates a side cross-sectional view of the assembled connection with the analyte sensor 1210. FIG. 11D illustrates a top view of the assembled connection.

The conductive tabs 1300 and configuration of the sensor receiving portion may be utilized with an implementation disclosed herein.

Any implementation disclosed herein may utilize such a sensor connection for an analyte sensor.

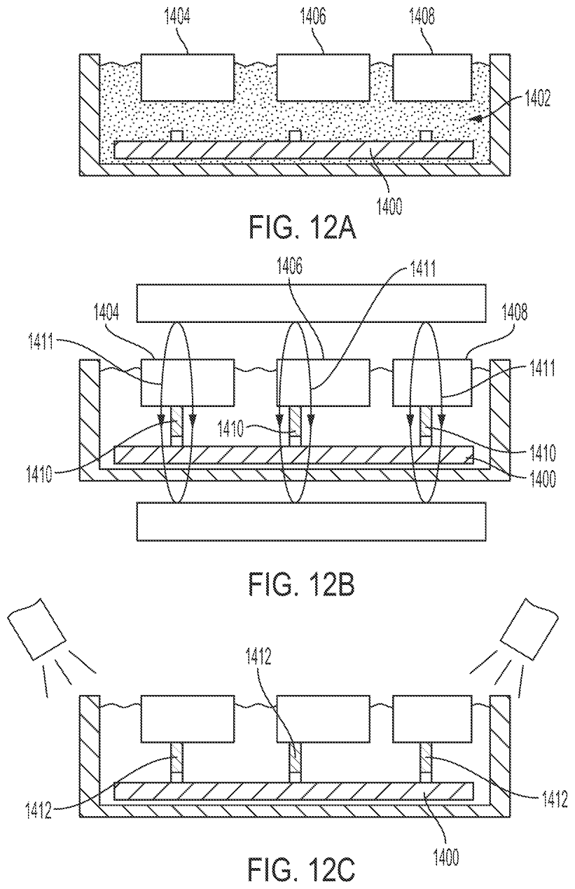

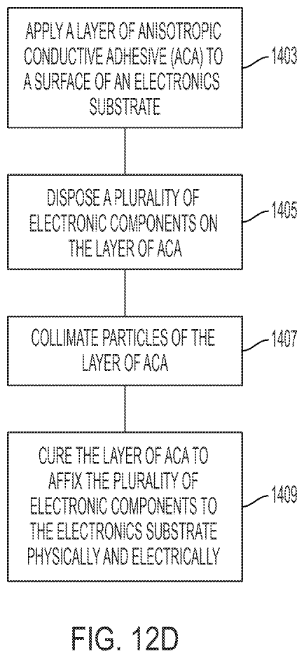

FIGS. 12A-12D illustrate a system and method for forming electrical conduits utilizing anisotropic conductive adhesives (ACAs). ACAs comprise materials that may be responsive to electric fields or magnetic fields, or both, to align and be placed in a desired position. The ACAs may be collimated and then cured to form electrical conduits. The ACAs may comprise ferritic or ferrous particles and may be suspended in a carrier material such as material that may be used for electrical encapsulation or potting.

Referring to FIG. 12A, an electrical substrate 1400 may be provided within a body such as a housing disclosed herein. Other forms of bodies may be utilized. The body may be configured to retain material such as ACAs and/or a carrier material.

The ACAs 1402 may be positioned on the electrical substrate 1400 as shown in FIG. 12A for example. The ferritic or ferrous particles may be floating within a carrier material in FIG. 12A. Step 1403 of FIG. 12D, for example, may illustrate such a step. The ACAs 1402 may be provided in an aerosol dispensing, a pad/screen printing, or direct-style volume dispense, among other forms of dispensing.

Other electrical components for the electrical conduits to connect with may be provided. The electrical components may comprise one or more of a battery 1404, sensor electronics 1406, or a socket 1408 for coupling to a sensor, among other forms of electrical components. Step 1405 of FIG. 12D, for example, may illustrate such a step.

The particles of the ACA may be collimated into columns 1410. Referring to FIG. 12B, such a process may include applying a magnetic field 1411 (as shown in FIG. 12B) or an electric field, or another process, to the ACA 1402 to collimate the ACA 1402. The pattern of the field (e.g., magnetic or electric field) may be controlled to produce a desired pattern of the electrical conduits. Step 1407 of FIG. 12D, for example, may illustrate such a step.

Referring to FIG. 12C, the ACA may be cured to produce the electrical conduits 1412. For example, heat or thermal curing, ultraviolet (UV) curing, or other forms of curing may be utilized. Step 1409 of FIG. 12D, for example, may illustrate such a step. Curing may couple or affix the electrical components to the electrical substrate 1400 physically and/or electrically. In implementations, curing may include utilizing a self-catalyzing material.

The resulting electrical conduits 1412 may have conductivity in a direction perpendicular to a plane of the electrical substrate 1400.

The processes may comprise filling a housing or body with ACA and then providing a desired pattern of electrical conduits. The carrier material remaining within the housing or body may be cured to form a seal of the interior of the housing or body.

The forms of electrical conduits that may be formed may include electrical conduits for transmitting electrical energy to or from the analyte sensor. The electrical energy may comprise an electrical signal to or from the analyte sensor. The electrical energy may comprise electrical power to or from the analyte sensor. An electrical component may comprise a battery 1404. The electrical conduits may transmit power from the battery to one or more electrical conduits positioned within the body or housing. An electrical component may comprise a wireless transmitter. An electrical component may comprise one or more electrical terminals for the analyte sensor. An electrical component may comprise sensor electronics. The electrical conduits may electrically connect the electrical components to an electrical substrate in the form of a printed circuit board (PCB).

ACAs may be batch applied and cured all at once to improve manufacturing efficiencies. All electrical conduits within a housing or body may be formed with ACAs according to implementations herein.

Any implementation disclosed herein may utilize ACAs or the methods of utilizing ACAs.

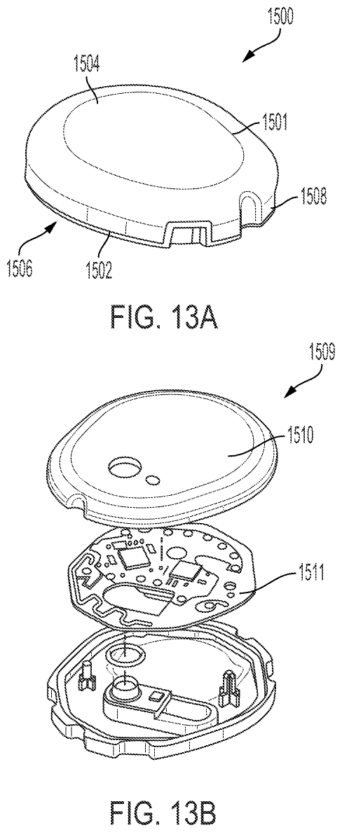

FIG. 13A illustrates a perspective view of an on-skin wearable medical device 1500 according to implementations herein. The device 1500 may comprise a body 1501 configured to be worn on the skin and configured to couple to a transcutaneous analyte sensor, in a manner as may be disclosed herein. The body 1501 may be configured as a housing. The body 1501 may be configured to retain one or more electrical components therein. The body 1501 may be configured to be placed adjacent to the skin of a host.

The body 1501 have a base 1502 and an enclosure 1504 coupled to the base 1502. The coupling may be facilitated via an adhesive in implementations, among other forms of coupling. In implementations, the coupling may create a seal between the base 1502 and the enclosure 1504. The seal may be air-tight and/or moisture-proof.

In some implementations, the adhesive may be a hotmelt adhesive or film including a thermoplastic (e.g., polyolefin). In some embodiments, the adhesive may be reactive polyurethane. A reactive polyurethane may be dispensed and moisture cured. In some embodiments, the adhesive may be silicone or epoxy. The silicone or epoxy may be cured using ultraviolet (UV) light to create a seal between the base 1502 and the enclosure 1504. Epoxy may be alternatively cured in an oven, which may be for about 30 minutes in an approximately 80 degrees Celsius temperature. Other forms of curing may be utilized. Similar to epoxy, acrylates may be used as an adhesive and cured under the same or similar conditions as epoxy using an oven. In some embodiments, a seal between the base 1502 and the enclosure 1504 may be achieved using welding. The welding techniques may include ultrasonic welding, laser welding, vibration welding, or electromagnetic welding.

At least a portion of the body 1501 may be made of a liquid crystal polymer (LCP). In implementations, for example, the enclosure 1504 may be entirely or partially made of a liquid crystal polymer (LCP). In other implementations, the enclosure 1504 may be completely or partially made of one or more of the following: polyproplene (PP), polyethylene terephthalate glycol (PETG), polycarbonate (PC), copolyester (CP), and cyclic olefin copolymer (COC). The enclosure 1504 may have low oxygen absorption and low moisture absorption. For example, the moisture absorption percentage may be between 0.01% and 0.06%. Preferably, the moisture absorption percentage may be 0.03%. Other amounts of absorption may be utilized. The enclosure 1504 may be biocompatible. The enclosure 1504 may be sterilizable. The enclosure 1504 may cover an entirety of the base 1502 such that the base 1502 is confined within the enclosure 1504.

The base 1502 may be entirely or partially made of PP, PETF, PC, CP, COC, or an LCP. The base 1502 may have a bottom surface 1506. The bottom surface 1506 may be facing away from the enclosure 1504. The enclosure 1504 may not extend over the bottom surface 1506. The bottom surface 1506 may be curved (e.g., concave) or flat. The bottom surface 1506 may be entirely or partially coupled to a patch (not shown). The patch may couple the body 1501 to the skin.

The base 1502 may retain one or more electrical components (for example as shown as electrical components 1511 in FIG. 13B) within a perimeter 1508 of the base 1502. The enclosure 1504 may extend over the electrical components. The one or more electrical components may include an electronics substrate (e.g., a printed circuit board, a flexible circuit board) and may include a power source, a transmitter, and/or a sensor electronically coupled to the electronics substrate. The electronic components may comprise sensor electronics. The power source (e.g., a battery, among other forms of power sources) may supply power to rest of the one or more electronic components.

The sensor may comprise an analyte sensor as disclosed herein, and may transcutaneously measure an analyte (e.g., glucose) in the blood of a user. The sensor may be configured to generate a signal indicative of an analyte concentration in a host. The sensor measurements may be communicated to the transmitter, which may then communicate the measurements to an output device. The body 1501 may retain one or more of the electrical components for receiving a signal from the analyte sensor. The output device may be a display, a computing device, or a portable electronic device by example. The user may view the measurements on a user interface of the output device and act based on the measurements (e.g., perform medical treatment, seek medical attention, consume food). The sensor may be configured to extend from the body 1501 to be positioned within the skin. The aforementioned features of the device 1500 discussed in this paragraph may be included in other on-skin wearable medical devices discussed throughout this disclosure.

FIG. 13B illustrates an exploded view of an on-skin wearable medical device 1509. The device may have the same specifications of the device of FIG. 13A, except the enclosure 1510 may be made of LCP entirely.

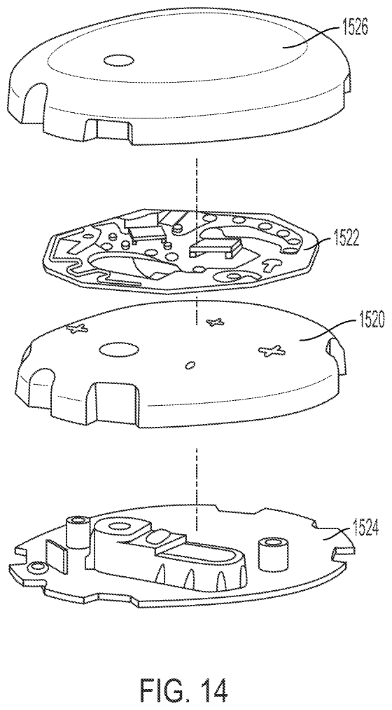

FIG. 14 illustrates an exploded view of an on-skin wearable medical device. The device may include one or more aspects of the device 1500 and may additionally include a filler 1520. In some implementations, the filler 1520 may be sandwiched between an electrical substrate 1522 and the base 1524. The electrical substrate 1522 in examples may include one or more electrical components such as a processor, a battery, and/or a memory storage. The filler 1520 may be sandwiched between the enclosure 1526 and the electrical substrate 1522. In some implementations, the filler may be positioned between a first housing and a second housing. The filler 1520, for example, may be disposed between the base 1524 and the enclosure 1526 or other forms of housings. The filler 1520 may occupy the empty space or voids between the enclosure 1526 and the base 1524. The filler 1520 may partially or completely surround electrical substrate 1522. The filler 1520 may comprise a thermoplastic and/or a thermoset polymer among other forms of material.

In some embodiments, filler 1520 may consist of, or include, a hotmelt material such as a thermoplastic polyamide or polyolefin. In such embodiments, the hotmelt material is configured to be molded at a low pressure, otherwise known as low pressure molding (LPM), which helps protect sensitive electronics and also cools or sets quickly to improve manufacturing throughput. The hotmelt material may cool or set more quickly than a thermoset polymer following molding. The hotmelt material may cool or set more quickly than the materials that the base and the enclosure are at least partially composed of following molding.

Enclosure 1526 and base 1524 may also be a molded component, but can be made using a different material or polymer using a different process. For example, enclosure 1526 and base 1524 can be molded from a thermoset polymer using conventional injection molding techniques, which occur at higher pressures and temperatures than an LPM process. In this manner, the polymers used to form enclosure 1526 and base 1524 can have different chemical and physical properties, such as improved hardness, improved cohesiveness, improved abrasion resistance, and/or reduced moisture permeability, when compared to a hotmelt material of an LPM process. The base 1524 and enclosure 1526 may be composed of a first material, and the hotmelt material may be configured to be molded at a lower pressure or temperature than the first material. The hotmelt material may comprise a filler composed of a different material than the base and/or enclosure. In some embodiments, enclosure 1526 and base 1524 can be molded from poly(methyl methacrylate) (PMMA), acrylonitrile butadiene styrene (ABS), nylon (polyamide, PA), polycarbonate (PC), polyethylene (PE), polyoxymethylene (POM), polypropylene (PP), polystyrene (PS), thermoplastic elastomer (TPE), thermoplastic polyurethane (TPU), and/or combinations thereof. The enclosure 1526 and base 1524 may be made of a different material or polymer than each other. In examples, the enclosure 1526 and base 1524 may be made of the same material.

In some embodiments, filler 1520 may consist of, or include, of a curable material. For example, the curable material can be a platinum-cured silicone, polyurethane, polysulfide, polyurea, epoxy, and combinations thereof. The curable material cures via an addition reaction, through the use of a catalyst, and/or a chemical reaction that does not require moisture.

In some embodiments, on-skin wearable medical device 1500 can be assembled or at least partially assembled according to the following method. In a first step, electrical substrate 1522 may be first placed either in enclosure 1526 or base 1524. Enclosure 1526 and/or base 1524 may include alignment and/or securement features configured to align and/or secure electrical substrate 1522. In a second step, the other of enclosure 1526 or base 1524 is assembled against the combined electrical substrate 1522 and enclosure 1526 or base 1524. In a third step, the assembled enclosure, 1526, base 1524, and electrical substrate 1522 is placed into a mold. In a fourth step, filler 1520 (e.g. hotmelt material) is injected into the cavity (e.g. via a LPM process) within the assembled enclosure 1526 and base 1524. In a fifth step, the filler 1520 is allowed to cool and set.

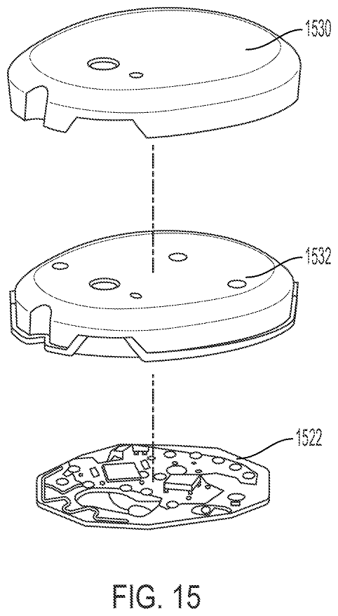

FIG. 15 illustrates an exploded view of an on-skin wearable medical device. The device may have an outer shell 1530, which may comprise a hardened outer shell 1530. The outer shell 1530 may comprise an outer shell 1530 of an enclosure and may extend over an inner shell 1532 of the enclosure. The outer shell 1530 may be made of LCP. Electronic components may be positioned within the housing (e.g., positioned upon the electrical substrate 1522). The inner shell 1532 may be made of one or more of a nylon plastic, a polyolefin, or a thermoplastic elastomer, among other materials. The outer shell 1530 may sit flush over the inner shell 1532. In implementations, the outer shell 1530 may be made of another material such as COC, or another material, by example.

Any implementation disclosed herein may include features of FIGS. 13A-15. Any implementation disclosed herein may utilize liquid crystal polymer (LCP) in a body, housing or other component as desired.

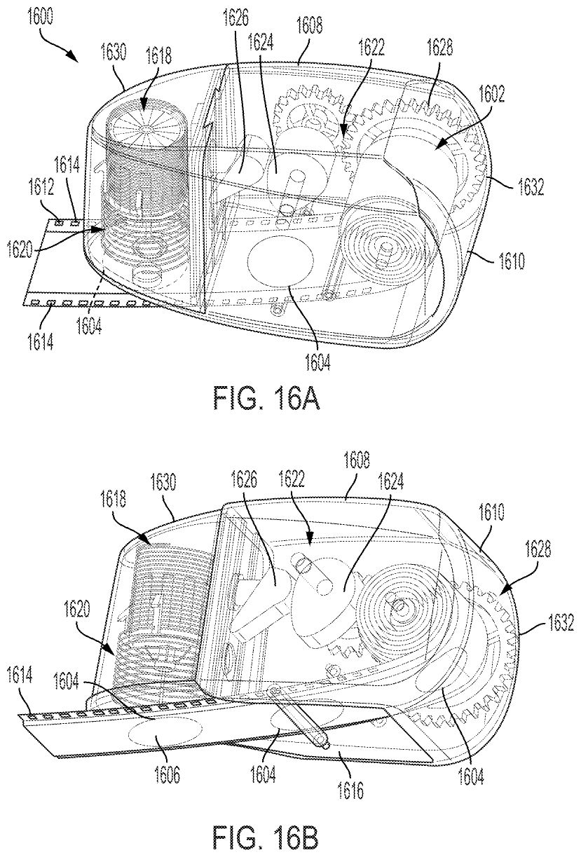

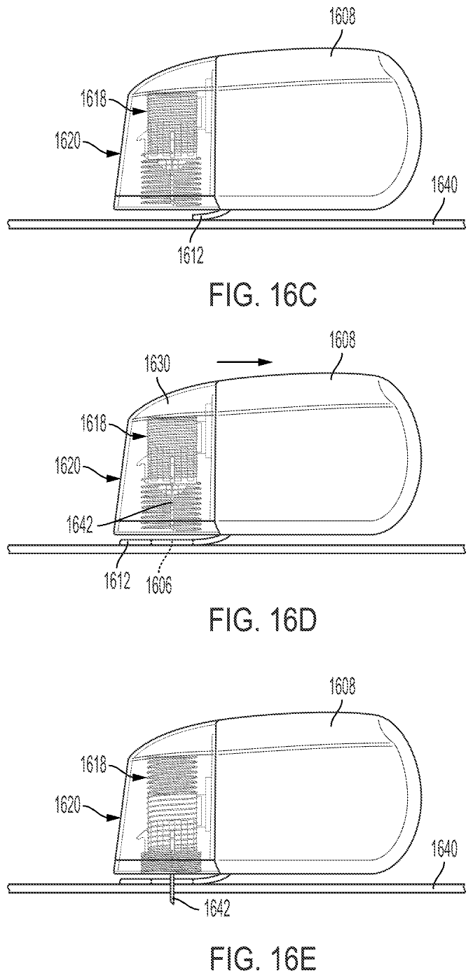

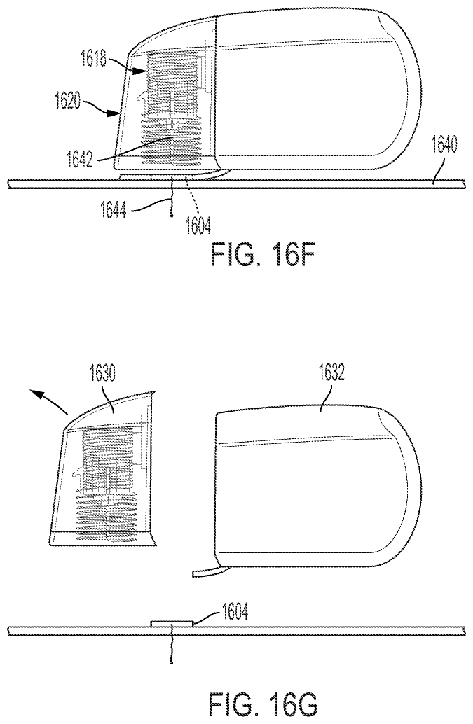

FIGS. 16A-16G illustrate an implementation of a system 1600 that may be utilized herein. Aspects of the system 1600 may comprise an on-skin wearable medical device or on-skin sensor assembly configured to be peeled onto a host's skin to at least partially deploy the on-skin wearable medical device or on-skin sensor assembly to the host's skin. Aspects of the system 1600 may include a reel 1602 configured to retain a plurality of flexible on-skin wearable medical devices or on-skin sensor assemblies to the reel 1602, the on-skin wearable medical devices or on-skin sensor assemblies configured to be deployed from the reel 1602 to a host's skin. Other aspects of the system are disclosed herein.

The system 1600 may include one or more on-skin wearable medical devices or on-skin sensor assemblies. All or a portion of the on-skin wearable medical devices or on-skin sensor assemblies may be represented by the housings 1604 shown in FIGS. 16A-16G. The housings 1604 may comprise flexible housings configured to flex, and may be capable of being rolled onto a reel 1602 or another device for retaining the housings 1604. At least a portion of the on-skin wearable medical device may be flexible.

In implementations, the housings 1604 may include sensor electronics, power supplies, sensor sockets, or a transcutaneous analyte sensor coupled thereto, although in implementations any or all of those components may be added to the housings 1604 during deployment or following deployment to the host's skin. For example, as shown in FIG. 16A, the housing 1604 may be deployed to the host's skin and then electronics or analyte sensors may be coupled thereto, or in implementations the housings 1604 may contain all components for operation. The on-skin wearable medical device may be configured to retain one or more electrical components for the on-skin wearable medical device. One or more of a battery, a transmitter, or contacts for a transcutaneous analyte sensor may be provided. The housings 1604 may contain certain components yet not all components for operation. For example, an analyte sensor in implementations may be coupled to the housings 1604 during deployment.

The housings 1604 may be configured to adhere to the host's skin. For example, the housings 1604 may include a patch 1606 (marked in FIG. 16B) or adhesive side that is configured to couple to the host's skin. In implementations, a liner may be positioned over the patch 1606 or adhesive side, to be removed prior to deployment.

Upon the patch or adhesive side being exposed, the housings 1604 may be peeled onto the host's skin to at least partially deploy the on-skin wearable medical device or on-skin sensor assembly to the host's skin. Such a peeling may comprise a wipe or wiping movement, or a lateral movement of the housings 1604 that may be parallel to the plane of the host's skin. The movement may be along the plane of the host's skin and may include a travel along the host's skin along the parallel plane. Other forms of peelings may be utilized. In implementations, only a patch may be peeled and not a housing, or an entire on-skin wearable medical device or on-skin sensor assembly may be peeled for deployment. The on-skin wearable medical device may be peeled onto the host's skin to insert a transcutaneous analyte sensor of the on-skin wearable medical device into the host's skin.

FIGS. 16A-16G illustrate an implementation of an applicator 1608 that may be utilized for peeling the on-skin wearable medical device or on-skin sensor assembly to the host's skin to at least partially deploy the on-skin wearable medical device or on-skin sensor assembly to the host's skin.

The applicator 1608 may include an applicator housing 1610 that may be configured to be gripped by a user. The applicator 1608 may retain the reel 1602 that may be configured to retain the on-skin wearable medical device or on-skin sensor assembly.

In implementations, an elongate retainer body 1612 may be provided that may be configured to retain the on-skin wearable medical device or on-skin sensor assembly and wrap about the reel 1602. The elongate retainer body 1612, for example, may comprise a ribbon that wraps about the reel 1602. The on-skin wearable medical device or on-skin sensor assembly, or housing 1604 of such a device or assembly may be releasably coupled to the elongate retainer body 1612 and configured to release from or peel from the retainer body 1612 at a desired time. The elongate retainer body 1612, for example, may include perforations or other releasable couplings between the on-skin wearable medical device or on-skin sensor assembly, or housing 1604, and the elongate retainer body 1612. In implementations, the elongate retainer body 1612 may include a track or registration guide 1614 that may be utilized for positioning of the elongate retainer body 1612 and to determine how much of the elongate retainer body 1612 has been drawn from the reel 1602.

The elongate retainer body 1612 may include one or more of the on-skin wearable medical devices or on-skin sensor assemblies, or housings 1604. The elongate retainer body 1612 may be configured to retain a plurality of the on-skin wearable medical devices.

The applicator housing 1610 may include a dispenser opening 1616 (marked in FIG. 16B) for the elongate retainer body 1612 and the on-skin wearable medical devices or on-skin sensor assemblies, or housings 1604, to be dispensed from. The dispenser opening 1616 may be positioned on a bottom portion of the applicator housing 1610 or another portion as desired. The dispenser opening 1616 may be configured for the elongate retainer body 1612 to be drawn from.

The dispenser opening 1616 may be positioned such that the elongate retainer body 1612 or adhesive portion of the housing 1604 may be contacted to the host's skin. The elongate retainer body 1612 may be drawn from the reel 1602 upon the applicator housing 1610 being moved laterally along the host's skin along the skin with an adhesive from the housing 1604 or another adhesive adhering to the skin. The elongate retainer body 1612 may be peeled along the host's skin and drawn from the applicator while being peeled along the host's skin.

The applicator 1608 may include an insertion assembly 1618 configured to insert a needle into the host's skin to insert the analyte sensor into the host's skin. The insertion assembly 1618, for example, may include an insertion driver such as an insertion spring configured to drive the needle into the host's skin along with the analyte sensor. The insertion assembly 1618 may be configured to be positioned above the on-skin wearable medical device or on-skin sensor assembly to insert the analyte sensor into the host's skin.

The applicator 1608 may include a retraction assembly 1620 in the form of a retraction driver, such as a retraction spring configured to retract the needle from the host's skin after the analyte sensor has been deployed to the skin. The analyte sensor may remain within the host's skin upon retraction of the needle. The retraction assembly 1620 may be configured to automatically retract the needle following insertion of the needle. The retraction assembly 1620 may retract the needle from the host's skin following insertion of the transcutaneous analyte sensor of one of the on-skin wearable medical devices into the host's skin with the needle.

The applicator 1608 may include a trigger assembly 1622 configured to activate the insertion assembly 1618 at a desired time. The trigger assembly 1622 may trigger the insertion of the needle into the host's skin. The trigger assembly 1622, for example, may include a cam 1624 and an anvil 1626 configured to be hit by the cam 1624 at a desired time to activate the insertion assembly 1618. The cam 1624 may be configured to rotate based on one or more gears 1628 that are rotated based on the elongate retainer body 1612 being drawn from the reel 1602. The track or registration guide 1614 may be coupled to the gears 1628 in implementations to cause the gears 1628 to rotate or another method may be utilized. The trigger assembly 1622 may be actuated based on movement of the applicator 1608 relative to the host's skin. The trigger assembly 1622 may insert the needle into the host's skin.

In implementations, the insertion assembly 1618 and retraction assembly 1620 and a needle utilized (whether prior to insertion, or a used needle produced following insertion) may be retained in a first portion 1630 of the applicator 1608 that may be separable from a second portion 1632 of the applicator 1608. The first portion 1630 may further include the analyte sensor in implementations. The second portion 1632 may retain the reel 1602 and the trigger assembly 1622. As such, the needle and the analyte sensor may be sterilized separately from the second portion 1632 as desired. The first portion 1630, for example, may include one or more ports for receiving a sterilizing gas or other form of sterilant.

Further, the first portion 1630 may be discarded following use if the first portion 1630 retains the used needle. The used needle may comprise a biohazard for disposal. The second portion 1632 may be reused as desired and may comprise a reusable applicator body. The first portion 1630 may retain the used needle following insertion into the host's skin.

FIGS. 16C-16G illustrate an exemplary deployment operation of the on-skin wearable medical device or on-skin sensor assembly. FIG. 16C illustrates the applicator 1608 may be slid laterally along the host's skin 1640 with an adhesive portion of the on-skin wearable medical device or on-skin sensor assembly, or an adhesive portion of the elongate retainer body 1612 contacting the skin. The adhesion may cause the elongate retainer body 1612 to be drawn from the reel 1602 shown in FIG. 16A. The on-skin wearable medical device or on-skin sensor assembly may be peeled onto the host's skin and from the applicator.

FIG. 16D illustrates the elongate retainer body 1612 having been drawn from the reel 1602 and the on-skin wearable medical device or on-skin sensor assembly, or housing 1604, may be positioned beneath the needle 1642. The trigger assembly 1622 shown in FIGS. 16A and B may be activated to activate the insertion assembly. In implementations, the analyte sensor may be positioned within the first portion 1630 for insertion into the host's skin.

FIG. 16E illustrates the insertion assembly 1618 having operated, with the needle 1642 and analyte sensor inserted into the host's skin 1640. FIG. 16F illustrates the retraction assembly 1620 having been actuated to retract the used needle 1642. The analyte sensor 1644 may remain within the host's skin 1640. The housing 1604 may then be separated from the elongate retainer body 1612 and may remain on the host's skin 1640. Electrical components may be added to the housing 1604 as desired.

Referring to FIG. 16G, the first portion 1630 may be separated from the second portion 1632 following use. The first portion 1630 may be disposed, and the second portion 1632 may be reusable, particularly if the second portion 1632 includes additional on-skin wearable medical devices or on-skin sensor assemblies, or housings 1604.

Variations in the systems disclosed herein may be provided.

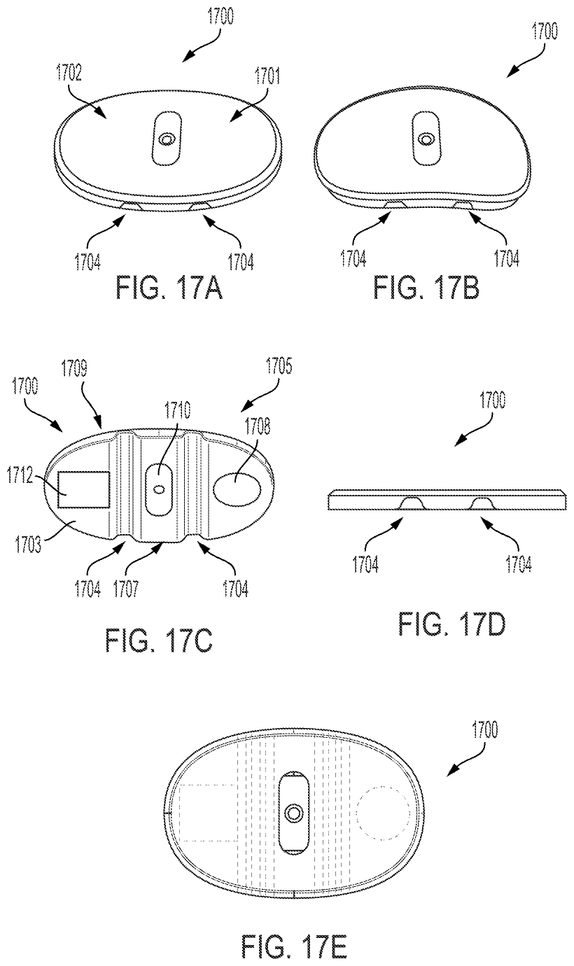

FIGS. 17A-25B illustrate implementations of bodies including one or more bending sections configured to allow the body to bend to conform to a contour of the skin.

FIGS. 17A-17E, for example, illustrate an implementation of a body 1700 in the form of a housing including an exterior 1701 and may include an interior (e.g., an interior cavity for receiving electrical components or other features of the body). The exterior 1701 may include a top surface 1702 and may include a bottom surface 1703 (marked in FIG. 17C).

The top surface 1702 or the bottom surface 1703 may include one or more bending sections in the form of one or more living hinges 1704. The living hinges 1704 may comprise channels in the top surface 1702 or the bottom surface 1703 of the body 1700 configured to allow the body 1700 to flex. The channels may extend along an exterior of the housing. Two living hinges 1704 are shown in FIGS. 17A-17E, yet a lesser or greater number may be provided as desired.

The living hinges 1704 may comprise channels extending along the top surface 1702 or the bottom surface 1703. The living hinges 1704 may have other configurations in other implementations.

In the implementation of FIGS. 17A-17E, the living hinges 1704 may be positioned on the bottom surface 1703 of the body 1700. The living hinges 1704 may extend along the short dimension or minor axis of the body 1700 in an implementation in which the body 1700 has an elongated or oblong configuration. The top surface 1702 of the body 1700 may comprise a smooth surface that may lack a living hinge.

In implementations, the living hinges 1704 may segment the body 1700 into rigid portions and flexible portions. Rigid portions, for example, may comprise the rigid portions 1705, 1707, 1709 (marked in FIG. 17D) and the flexible portions may comprise the bending sections or sections including the living hinges 1704.

Referring to FIG. 17C, in implementations, electrical components may be positioned within the rigid portions 1705, 1707, 1709. For example, a power source or battery 1708 may be positioned in a rigid portion 1705, a sensor coupler 1710 may be positioned in a rigid portion 1707, and sensor electronics 1712 may be positioned in a rigid portion 1709. Flexible electrical conduits or a flexible electrical substrate may join the electrical components though the bending sections. A flexible circuit board may be utilized. The flexible circuit board may be disposed in an interior of the body and configured to be coupled to one or more electrical components and bend with the body. The electrical components may remain unflexed in the rigid portions while the bending sections are flexed.

The bending sections may allow for bending in a direction. In the implementations of FIGS. 17A-17E, the body 1700 may be configured to bend in a direction towards the host's skin or away from the top surface 1702 (as shown in FIG. 17B for example). In implementations, bending may occur in a direction away from the host's skin or towards the top surface 1702 (e.g., away from the bottom surface) as well.

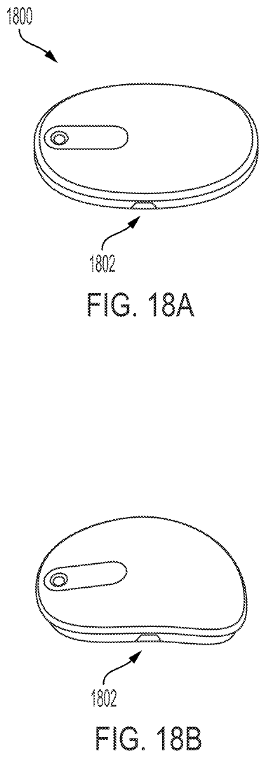

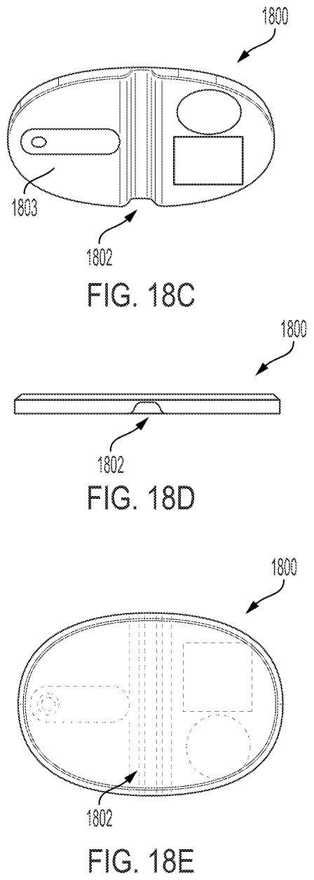

FIGS. 18A-18E illustrate an implementation of a body 1800 in the form of a housing including a single bending portion. The bending portion may comprise a living hinge 1802 that may be positioned on a bottom surface 1803 of the body 1800. The living hinge 1802 may extend along a short dimension or minor axis of the body 1800 in an implementation in which the body 1800 has an elongated or oblong configuration. The living hinge 1802 may extend along the minor axis central to the body 1800 or housing.

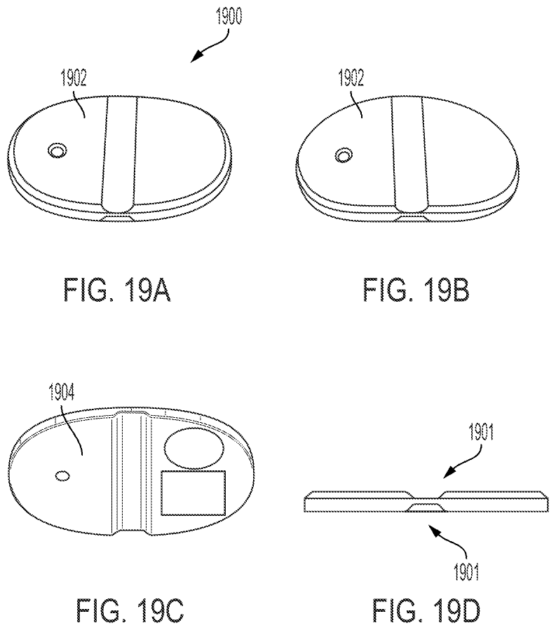

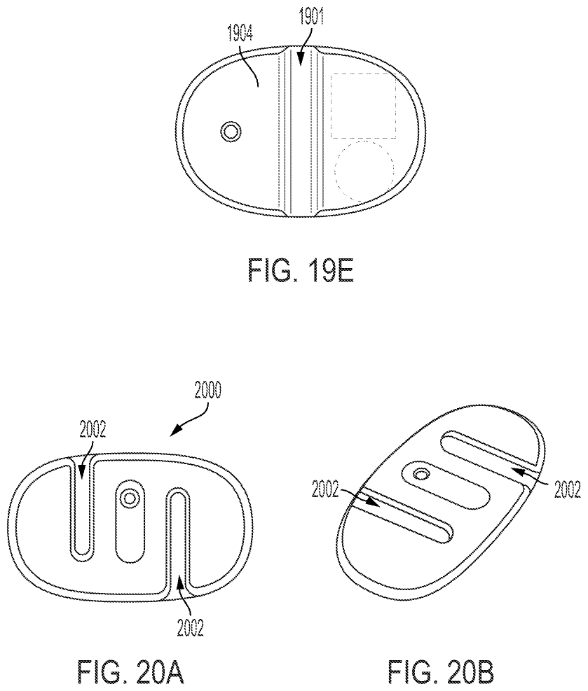

FIGS. 19A-19E illustrate an implementation of a body 1900 in the form of a housing including a bending portion in the form of living hinges 1901 on both a top surface 1902 and a bottom surface 1904 of the body 1900. Improved flexibility in a direction towards the host's skin and away from the host's skin may be provided.

FIGS. 20A-20B illustrate an implementation of a body 2000 in the form of a housing having an “S” shape, with concavities 2002 forming the bending sections and forming the “S” shape. The concavities 2002 may form cut-outs in the body 2000. The body 2000 may be configured to flex about the bending sections. The “S” shape may conform to an elliptical perimeter or may have another shape as desired. Each of the bending sections may include a concavity forming the “S” shape or S-shape.

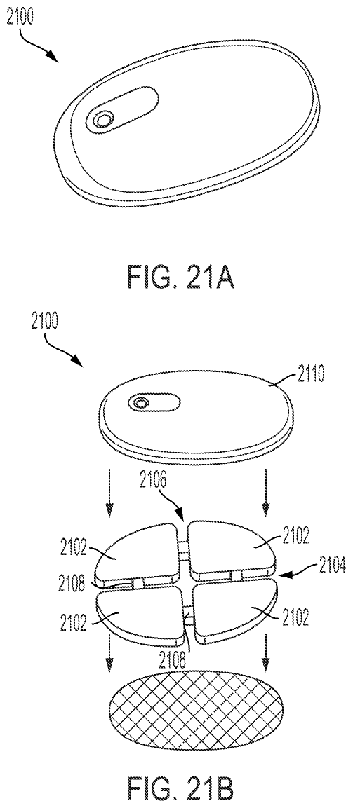

FIGS. 21A-21B illustrate an implementation of a body 2100 in the form of a housing having a plurality of compartments 2102. The compartments 2102 may be separated by bending sections 2104, 2106 configured to allow the body 2100 to flex. The bending section 2104, for example, may comprise a horizontal living hinge, and the bending section 2106 may comprise a vertical living hinge. The compartments 2102 may be separated by the living hinges and able to flex about both bending sections 2104, 2106. In implementations, electrical conduits or an electrical substrate may pass through flexible connections 2108 between the compartments 2102.

In implementations, a cover 2110 may extend over the compartments 2102. The cover 2110 may be configured to flex with the compartments 2102 and the remainder of the housing. The cover may be flexible and configured to bend with the housing.

The body 2100 may have an elliptical or elongate configuration in implementations, or another configuration as desired.

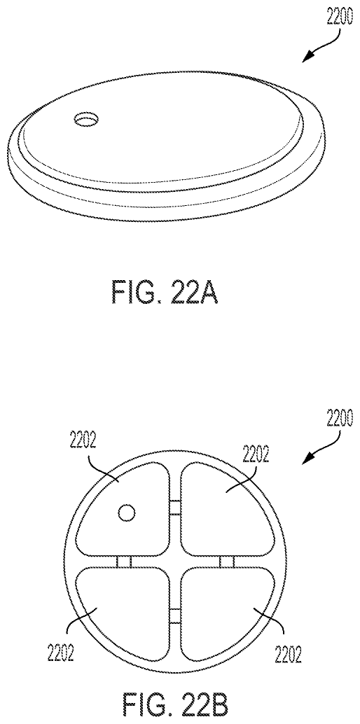

FIGS. 22A-22B illustrate an implementation of a body 2200 configured similarly as the body 2100 shown in FIGS. 21A-21B, yet having a circular outer profile. The body 2200 may include compartments 2202 separated by bending sections configured to allow the body 2200 to flex.

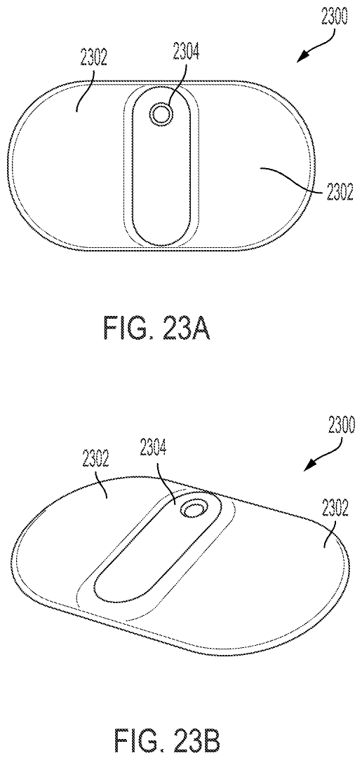

FIGS. 23A-23B illustrate an implementation of a body 2300 having bending sections in the form of flexible wings 2302. The wings 2302 may extend outward from a housing 2304 that may be rigid or may be flexible. The wings 2302 may comprise a cloth material or elastomeric material or other form of flexible material.

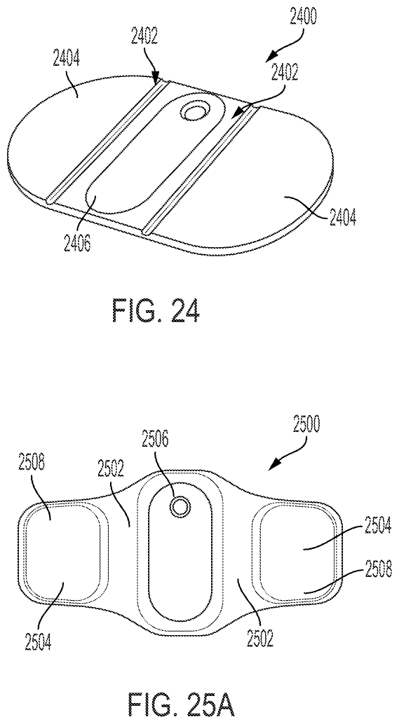

FIG. 24 illustrates an implementation of a body 2400 including bending sections in the form of living hinges 2402 configured to allow wings 2404 to flex relative to a central portion 2406. The central portion 2406 and the wings 2404 may be flexible or rigid as desired.

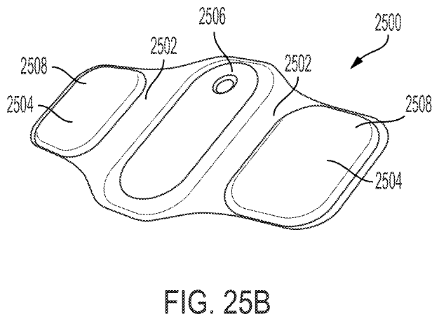

FIGS. 25A and 25B illustrate an implementation of a body 2500 including living hinges 2502 in the form of cloth extending between rigid portions or compartments 2504. The rigid portions or compartments 2504 may comprise a central compartment 2506 and peripheral compartments 2508.

Any implementation of body or housing disclosed herein may include one or more bending sections.

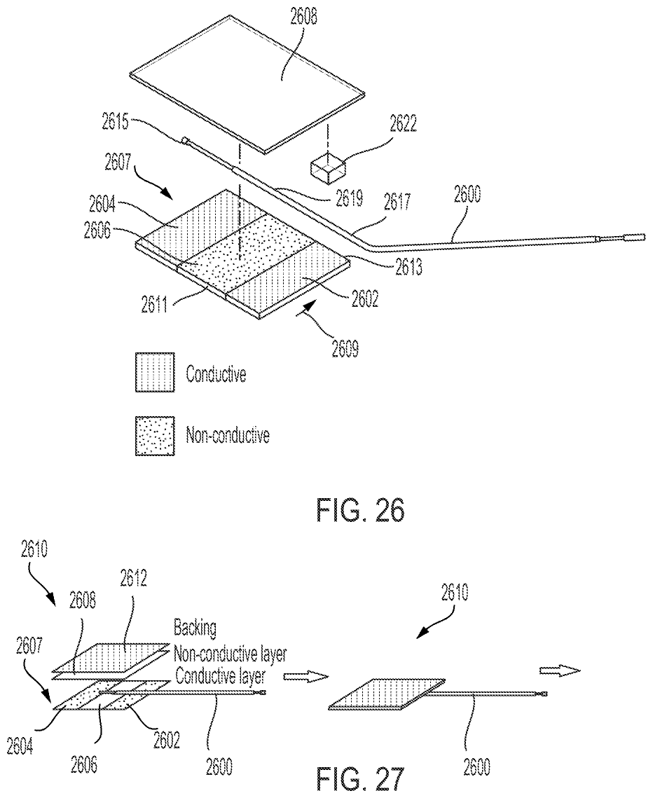

FIG. 26 illustrates an implementation of a construction of a sensor connection. The sensor connection may comprise a sensor carrier or sensor interposer as desired. The sensor connection may include the analyte sensor 2600, and a first conductive film 2602 and a second conductive film 2604. A non-conductive film 2606 may be laterally sandwiched by the first conductive film 2602 and the second conductive film 2604. The second conductive film 2604 may be disposed next to the first conductive film 2602.

The first conductive film 2602, the second conductive film 2604, and the non-conductive film 2606 may each comprise films delivered from a roll and may be thermoformed to form thin films that are disposed adjacent to each other. The films 2602, 2604, 2606 may be drawn out in a width-wise direction 2609 shown in FIG. 26 adjacent to each other and then cut or otherwise singulated to form edges 2611, 2613. The film 2602, 2604, 2606 may comprise a triblock configuration.

The first conductive film 2602 and the second conductive film 2604 may each comprise a conductive material. Such conductive material may comprise a polymer which may be mixed with a conductive material. Such polymers may comprise an elastomeric polymer such as ethylene-vinyl acetate (EVA) or another form of polymer. The polymer may be mixed with a conductive material such as conductive carbon or silver, or nickel, or another form of conductive material. The film material may drawn out from a roll in the width-wise direction 2609 shown in FIG. 26.

The non-conductive film 2606 may comprise an insulator polymer material or another form of material. The non-conductive film 2606 may comprise a polymer such as ethylene-vinyl acetate (EVA) or thermoplastic polyurethane (TPU) or another form of non-conductive polymer. The non-conductive film 2606 may comprise a di-electric material positioned between the first conductive film 2602 and the second conductive film 2604.

The analyte sensor 2600 may be positioned upon the substrate 2607 with a working electrode 2615 positioned over the second conductive film 2604 and the reference electrode 2617 positioned over the first conductive film 2602. The analyte sensor 2600 may be disposed over the first conductive film 2602, the second conductive film 2604, and in electrical contact with the first conductive film 2602 and the second conductive film 2604. The analyte sensor 2600 may further comprise a non-conductive or insulator portion 2619 that may be positioned between the working electrode 2615 and the reference electrode 2617. The non-conductive or insulator portion 2619 may be positioned upon the non-conductive film 2606. In implementations, the configuration of the analyte sensor 2600 or the substrate 2607 may be varied. For example, the relative positions of the working electrode 2615 and reference electrode 2617 may be alternated, or the configuration of the non-conductive or insulator portion 2619 may be varied as desired.

The analyte sensor 2600 may be positioned upon the substrate 2607 during a process in which the films 2602, 2604, 2606 are being drawn out in the width-wise direction 2609. The analyte sensor 2600 may be laid upon the substrate 2607, and multiple analyte sensors may be laid upon adjacent substrates as the substrate is being drawn from a roll.

The analyte sensor 2600 may be adhered to the substrate 2607 utilizing a tack adhesive or another form of coupling. A tack adhesive, for example, may comprise silver epoxy or another form of adhesive. In implementations, the analyte sensor 2600 may be positioned on the substrate 2607 without use of an adhesive. The first conductive film 2602 and second conductive film 2604 may couple to a tip of the analyte sensor 2600. The tip may be configured to be disposed over the first conductive film 2602. The first conductive film 2602 may comprise a working electrode and the second conductive film 2604 may comprise a reference electrode.

Referring to FIG. 26, in implementations, a barrier film 2608 may be disposed over the analyte sensor 2600 and may be disposed over the films 2602, 2604, 2606. The barrier film 2608 may be disposed over the analyte sensor 2600 and films 2602, 2604, 2606 in an elongate sheet that is laid over the analyte sensor 2600 and the films 2602, 2604, 2606 in the width-wise direction 2609 as the films 2602, 2604, 2606 are drawn from a roll. The films 2602, 2604, 2606 and barrier film 2608 accordingly may be drawn out in long sheets with the lengths of the films 2602, 2604, 2606 and the barrier film 2608 aligned.

The barrier film 2608 may comprise a non-conductive material and may comprise a polymer. The barrier film 2608, for example, may comprise a non-conductive EVA or a thermoplastic such as low melting point thermoplastic (TPU), or polyolefin, or polyethylene terephthalate (PET), or polybutylene terephthalate (PBT). Other forms of non-conductive material may be utilized. The barrier film 2608 may be configured to be laminated upon the films 2602, 2604, 2606 and the analyte sensor 2600, which may be in a vacuum assisted process.

The barrier film 2608 may be configured to have moisture barrier properties and may seal the electrical connection between the analyte sensor 2600 and the films 2602, 2604. The barrier film 2608 may have reflow properties and may have adhesive properties to adhere to a sensor receiving portion of a body or housing for receiving the analyte sensor 2600. The barrier film 2608 may be cured to the films 2602, 2604, 2606 and the analyte sensor 2600. The barrier film 2608 may be disposed over the analyte sensor 2600 to create a seal over the analyte sensor 2600, the first conductive film 2602, the second conductive film 2604, and the non-conductive film 2606.

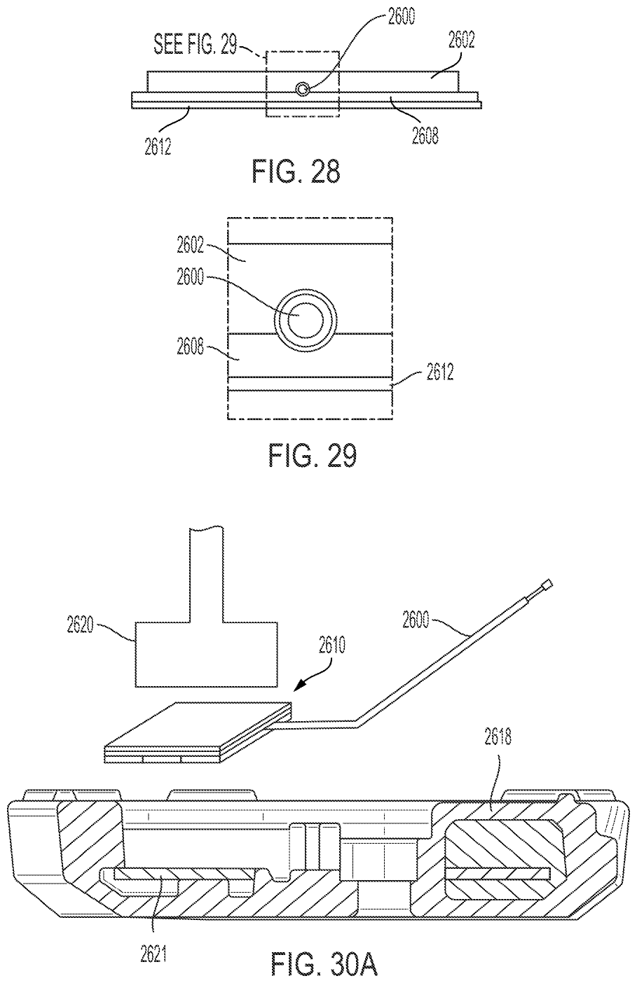

FIG. 27, for example, illustrates an assembly process in which the layers of films 2602, 2604, 2606, 2608 may be rolled together to form an assembly 2610. In implementations, a hardened backing layer 2612 may be disposed over the barrier film 2608. The hardened backing layer 2612 may be configured to cover and protect the films 2602, 2604, 2606, 2608. The backing layer 2612 may comprise a polymer material such as PET or another form of material. FIG. 27 illustrates the formed assembly 2610. The analyte sensor 2600 may extend from the films 2602, 2604, 2606 and may be configured to bend at a region away from the films 2602, 2604, 2606. FIG. 28 illustrates a cross-sectional assembled view of the formed assembly 2610 and FIG. 29 illustrates a detail view of the cross-sectional assembled view of FIG. 28.

In implementations in which the assembly 2610 is formed from a long continuous sheet of adjacent assemblies, the assembly 2160 may be singulated to form the edges 2611, 2613. The singulation may include cutting the assembly 2610 from the long sheet, which may include a laser or die cut or another form of cutting. In implementations, the films 2602, 2604, 2606 may be singulated prior to the barrier film 2608 or hardened backing layer 2612 being positioned thereon.

The assembly 2610 may comprise a sensor connect assembly or sensor carrier or interposer, and may be formed in a scaled sensor construction operation. Reel-to-reel processes may be utilized to combine the respective films and form the assembly 2610.

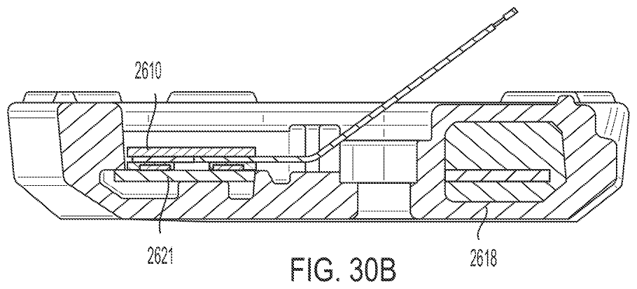

The assembly 2610 may be coupled to an electrical substrate. The electrical substrate may comprise an electrical substrate 2621 of a body or housing. The electrical substrate 2621 may connect to electrical components as may be disclosed herein. FIG. 30A, for example, illustrates a manufacturing step in which a heated press 2620 may be utilized to apply the assembly 2610 to a housing 2618 for the analyte sensor 2600. FIG. 30B illustrates the assembly 2610 coupled to the housing 2618. The assembly 2610 may be positioned within a sensor receiving portion in the form of a cavity of the housing 2618. Electrical connections may be formed between the assembly 2610 and the electrical substrate 2621. The first conductive film 2602, the second conductive film 2604, and the non-conductive film 2606 may be configured to couple to the electrical substrate 2621 to form an electrical connection between the analyte sensor 2600 and the electrical substrate.

In implementations, the barrier film 2608 may be reflowed within the cavity of the housing 2618 to seal the assembly 2610 within the cavity and seal the electrical connection between the assembly 2610 and the electrical substrate 2621. The barrier film 2608, for example, may be heated or otherwise caused to reflow to fill the cavity and encapsulate and protect the electrical connection. Various other methods of coupling or bonding may be utilized, such as pressing, curing, thermal bonding, tacking, roller heating, laser heating, or ultrasound, among others. The backing layer 2612 may cover the electrical connections as well. Referring to FIG. 26, a retainer 2622 may be provided that may further couple the sensor 2600 to the sensor receiving portion of the housing.

The assembly 2610 may be applied to the housing 2618 in other methods as desired.

Any implementation disclosed herein may utilize a sensor connection as discussed in regard to FIGS. 26-30B.

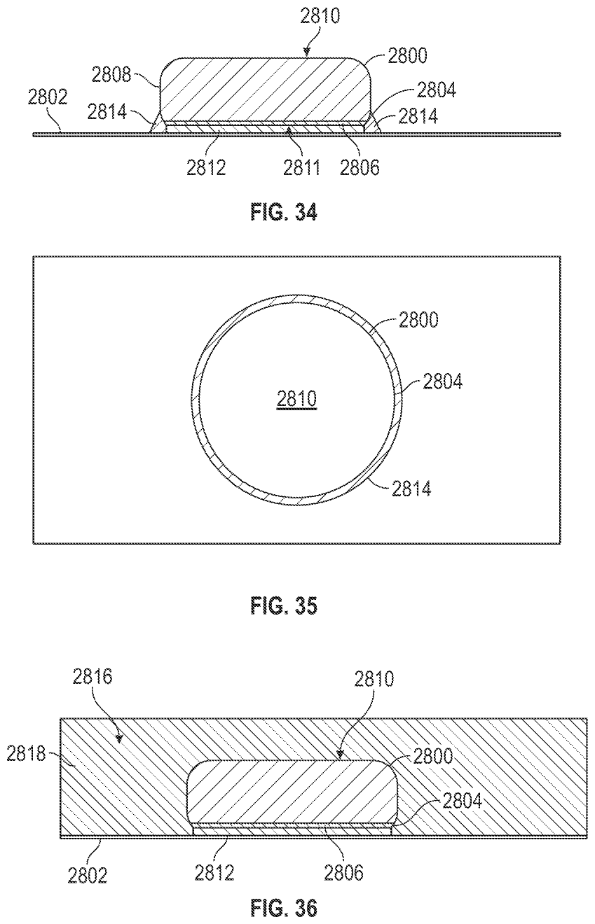

FIGS. 34-36 illustrate implementations in which a seal may be positioned at a perimeter of a battery to reduce moisture ingress to an electrical terminal of the battery. For example, referring to FIG. 34, a battery 2800 is illustrated positioned upon an electrical substrate 2802. The battery 2800 and/or electrical substrate 2802 may be positioned within a body as disclosed herein. For example, the body may comprise a housing having an interior cavity. The body may be configured to be worn on skin and configured to couple to a transcutaneous analyte sensor. Other forms of bodies may be utilized as desired.

The battery 2800 may have a perimeter 2804 and may include an electrical terminal 2806. The electrical terminal 2806 may be configured to provide for flow of electrical energy to other elements in contact with the electrical terminal 2806. The electrical terminal 2806 may comprise a negative terminal or a positive terminal. The battery 2800 may include an additional electrical terminal 2808 (a complementary positive or negative terminal) for completion of a circuit for the battery 2800 to provide electrical energy. The battery 2800 may be configured to provide power to one or more electrical components of an on-skin wearable medical device.

In implementations, the battery 2800 may comprise a coin-cell battery and the electrical terminal 2806 may be positioned on a lower surface 2811 or face of the battery 2800. Another electrical terminal 2808 may comprise a side wall or upper surface 2810 or face of the battery 2800. The battery 2800 may have a circular perimeter 2804, or another shape in implementations (e.g., rectangular, triangular, etc.).

The electrical terminal 2806 may be positioned upon the electrical substrate 2802. The electrical substrate 2802, for example, may include an electrical contact 2812 that may contact the electrical terminal 2806 for flow of electrical energy. The electrical contact 2812 may be positioned beneath the lower surface 2811 or face of the battery 2800.

It may be beneficial to reduce moisture ingress to the electrical terminal 2806. A seal 2814 may be positioned at the perimeter 2804 of the battery 2800 and may be configured to reduce moisture ingress to the electrical terminal 2806. The seal 2814 may reduce the possibility of moisture ingress beneath the lower surface 2811 or face of the battery 2800 and with a contact between the electrical terminal 2806 and the electrical contact 2812. As such, reduced possibility of wear, leakage, electrical damage, corrosion, a hydrolysis reaction, or electrical shorting may result.

Referring to FIG. 34, the seal 2814 may have a variety of forms. The seal 2814 may comprise a bead of material that may be positioned at the perimeter 2804 of the battery 2800. The seal 2814 may extend about the entirety of the perimeter 2804 of the battery 2800 as shown in the top view of FIG. 35. The seal 2814 may be positioned upon the electrical substrate 2802 and in contact with a surface of the electrical substrate 2802 and a surface of the battery 2800. The seal 2814 may serve to adhere or bond the battery 2800 to the electrical substrate 2802 in implementations.

The seal 2814 may comprise a curable material or foam or may have another configuration in implementations. The curable material may comprise an epoxy, resin, or adhesive, or other form of material configured to cure. The curable material may be dispensed in a liquid or fluid form and cured to a hardened state to seal the electrical terminal 2806. A foam may comprise a closed cell foam or other material configured to impede moisture transmission. Other forms of materials may be utilized as desired.

Seal materials that are high contrast or fluorescent may further be utilized to confirm a continuous seal upon visual inspection and sufficient sealing of the battery-substrate gap.

Other configurations of seals may be utilized. For example, referring to FIG. 36, an interior cavity 2816 of a housing may be filled with a curable material 2818 that forms the seal. The seal may cover the upper surface 2810 of the battery 2800 and the side surfaces or side wall of the battery 2800. A reduced possibility of moisture ingress to the entirety of the battery 2800 and the electrical connection with the electrical substrate 2802 may result. Curable material may comprise ultraviolet (UV) curable material or two-part epoxy, among other forms.

In assembly, the battery 2800 may be placed in position upon the substrate. The seal may be dispensed to the perimeter of the battery 2800. For example, a bead of material or a flood of material within the housing may be provided. A curing process may be applied. For example, a UV flood cure or other means for curing may be provided. In an implementation where material such as foam is utilized, the curing step may be excluded.

The features of FIGS. 34-36 may be utilized with any implementation as desired.

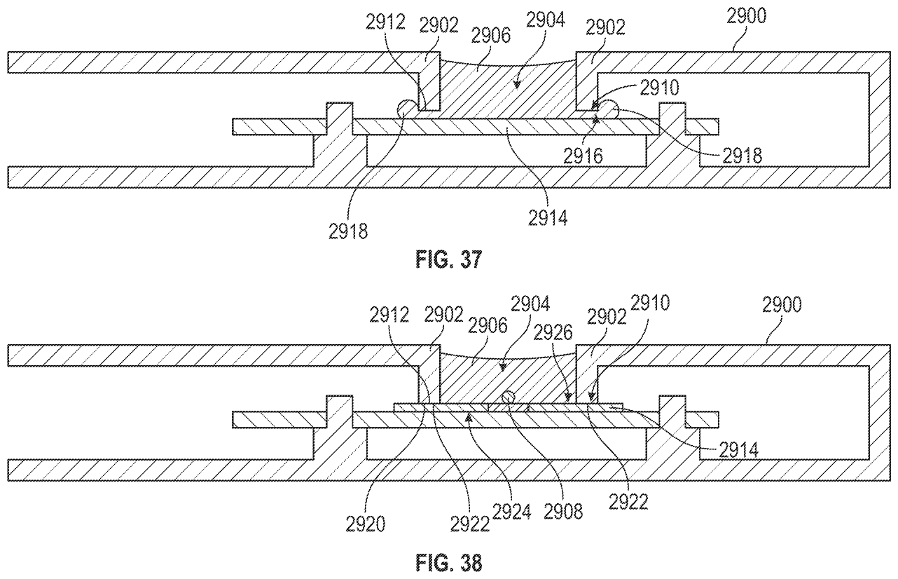

FIG. 38 illustrates an implementation in which an adhesive layer may be configured to impede curable material from egressing a cavity. For example, referring to FIG. 37, an implementation excluding use of such an adhesive layer is shown. A housing 2900 is illustrated. The housing 2900 may be configured similarly as any implementation of housing disclosed herein, or may have other forms. The housing 2900 may be configured to be worn on skin. The housing 2900 may be configured to couple to a transcutaneous analyte sensor.

The housing may include one or more walls 2902 defining a cavity 2904 for receiving a curable material 2906. The cavity 2904 may comprise a sensor receiving portion of a housing, similar in construction to the sensor receiving portions illustrated in FIGS. 9A-9B or 32A-32B. The cavity 2904 may be configured to receive an analyte sensor 2908 (marked in FIG. 38). The cavity 2904 may be utilized in other manners in implementations.

The cavity 2904 may have an end portion 2910 with an opening 2912. A substrate 2914 may be positioned within the housing 2900 and may be positioned at the opening 2912 of the cavity 2904. The substrate 2914 may have a variety of forms in implementations. The substrate 2914, for example, may comprise an electrical substrate that may be configured to electrically couple with the analyte sensor 2908. The substrate 2914 in implementations may be configured to support the analyte sensor 2908.

A gap 2916 or break may be positioned between the substrate 2914 and the walls 2902. Such a gap 2916 or break may result from the substrate 2914 comprising separate material as the walls 2902. The gap 2916 or break may be narrow or small in size, yet may be of sufficient size to allow for egress of the curable material 2906 from the cavity 2904.

The curable material 2906 may have a variety of forms and may comprise an epoxy, resin, adhesive or another form of curable material. The curable material 2906 may be electrically conductive in implementations. The curable material 2906 may be utilized for stabilization or sealing of the analyte sensor 2908 within the cavity 2904.

The curable material 2906 may be dispensed into the cavity 2904 in a fluid or liquid form, which may occur prior to the curing of the curable material 2906. FIG. 37, for example, illustrates the curable material 2906 dispensed into the cavity 2904 prior to full curing or hardening of the curable material 2906.

Referring to FIG. 37, the curable material 2906 may egress the cavity 2904 and may pass through the gap 2916 or break between the walls 2902 and the substrate 2914. FIG. 37, for example, illustrates portions 2918 of the curable material 2906 extending outward to other portions of the substrate 2914 that are outside of the opening 2912. Such a result may be undesirable, as the curable material 2906 may not be positioned in the cavity 2904 to a desired amount (due to the egress), and the curable material 2906 may be undesired on the other parts of the substrate 2914 or within the housing 2900. It may be difficult to maintain a desired or uniform thickness of the curable material 2906.

FIG. 38 illustrates an implementation in which an adhesive layer 2920 is positioned between the substrate 2914 and the opening 2912 of the cavity 2904. The adhesive layer 2920 may be configured to impede the curable material 2906 from egressing the cavity 2904 through the opening 2912. The curable material 2906 may be impeded from flowing through the opening 2912 onto a portion of the substrate 2914 that is positioned outward of the opening 2912.

The adhesive layer 2920, for example, may contact the ends 2922 of the walls 2902 that define the opening 2912 of the cavity 2904. The adhesive layer 2920 may contact the ends 2922 and the substrate 2914 to reduce flow of the curable material 2906 therethrough.

In implementations, the adhesive layer 2920 may include a lower surface 2924 or first surface that may face and contact the substrate 2914. The adhesive layer 2920 may include an upper surface 2926 or second surface that may face opposite the lower surface 2924. Each surface 2924, 2926 may include an adhesive in implementations. As such, adhesion to the substrate 2914 and the walls 2902 may result.

The adhesive layer 2920 may comprise a pressure sensitive adhesive in examples. The adhesive layer 2920 may comprise a tape or may have another configuration in examples. The adhesive on the adhesive layer 2920 may comprise a tacky material that allows for adhesion to the substrate 2914 and/or the walls 2902. The adhesive layer 2920 may comprise a thin, conformal layer of pressure sensitive film.

In implementations, the adhesive layer 2920 may be electrically conductive. For example, referring to FIG. 38, the analyte sensor 2908 may be positioned upon and in contact with the adhesive layer 2920. The adhesive layer 2920 may be configured to electrically couple to the analyte sensor 2908. The adhesive layer 2920 may be configured to conduct electrical energy to the substrate 2914, which may be configured to electrically couple with the analyte sensor 2908.

In implementations, the adhesive layer 2920 may be non-conductive, may be utilized to retain the curable material within the cavity 2904 as desired.

The features of FIG. 38 may be utilized with any implementation as desired.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and ‘one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1.-75. (canceled)

76. A medical system comprising:
an on-skin wearable medical device configured to be peeled onto a host's skin to at least partially deploy the on-skin wearable medical device to the host's skin.

77. The medical system of claim 76, further comprising an applicator configured to at least partially deploy the on-skin wearable medical device to the host's skin.

78. The medical system of claim 77, wherein the applicator includes a reel configured to retain the on-skin wearable medical device.

79. The medical system of claim 78, further comprising an elongate retainer body configured to retain the on-skin wearable medical device and wrap about the reel.

80. The medical system of claim 79, wherein the on-skin wearable medical device is configured to release from the elongate retainer body to at least partially deploy to the host's skin.

81. The medical system of claim 79, wherein the elongate retainer body comprises a ribbon.

82. The medical system of claim 79, wherein the elongate retainer body is configured to be drawn from the reel.

83. The medical system of claim 79, wherein the elongate retainer body is configured to retain a plurality of on-skin wearable medical devices.

84. The medical system of claim 79, wherein the elongate retainer body is configured to be peeled along the host's skin and drawn from the applicator while being peeled along the host's skin.

85. The medical system of claim 77, wherein the on-skin wearable medical device includes a transcutaneous analyte sensor, and the applicator includes a needle configured to insert the transcutaneous analyte sensor into the host's skin.

86. The medical system of claim 85, wherein the applicator is configured to retract the needle from the host's skin.

87. The medical system of claim 85, further comprising a trigger assembly for inserting the needle into the host's skin.

88. The medical system of claim 87, wherein the trigger assembly is actuated based on movement of the applicator relative to the host's skin.

89. The medical system of claim 77, wherein the applicator includes a first portion and a second portion, the first portion configured to retain a used needle following insertion into the host's skin and the second portion retaining a reel configured to retain the on-skin wearable medical device, the first portion being separable from the second portion.

90. The medical system of claim 77, wherein the applicator includes an insertion assembly configured to be positioned above the on-skin wearable medical device to insert a transcutaneous analyte sensor of the on-skin wearable medical device into the host's skin.

91. The medical system of claim 76, wherein at least a portion of the on-skin wearable medical device is flexible.

92. The medical system of claim 76, wherein a flexible housing of the on-skin wearable medical device is configured to be peeled onto the host's skin.

93. The medical system of claim 76, wherein the on-skin wearable medical device is configured to be peeled onto the host's skin to insert a transcutaneous analyte sensor of the on-skin wearable medical device into the host's skin.

94. The medical system of claim 76, wherein the on-skin wearable medical device is configured to retain one or more electrical components for the on-skin wearable medical device.

95. The medical system of claim 94, wherein the one or more electrical components include one or more of a battery, a transmitter, or contacts for a transcutaneous analyte sensor.

96.-210. (canceled)
